(19) United States
(12) Patent Application Publication
(10) Pub. No.: US 2023/0030960 A1
(43) Pub. Date: Feb. 2, 2023

(54) A MOLDING COMPRISING A TI-MWW ZEOLITE AND HAVING A SPECIFIC LEWIS ACIDITY

(71) Applicant: BASF SE, Ludwigshafen am Rhein (DE)

(72) Inventors: Andrei-Nicolae PARVULESCU, Ludwigshafen am Rhein (DE); Ulrich MUELLER, Neustadt (DE); Jaroslaw Michael MORMUL, Ludwigshafen am Rhein (DE); Joaquim Henrique TELES, Ludwigshafen am Rhein (DE); Dominic RIEDEL, Ludwigshafen am Rhein (DE); Markus WEBER, Ludwigshafen am Rhein (DE)

(21) Appl. No.: 17/785,966

(22) PCT Filed: Dec. 18, 2020
(86) PCT No.: PCT/EP2020/087090

§ 371 Date: Jun. 16, 2022

(30) Foreign Application Priority Data
Dec. 20, 2019 (EP) 19218842.3

(51) Int. Cl. B01J 29/70; B01J 29/89; B01J 37/02; C07D 301/12; B01J 35/10

(52) U.S. Cl. CPC B01J 29/7088; B01J 29/89; B01J 37/0205; C07D 301/12; B01J 35/1042

(57) ABSTRACT

The present invention relates to a molding comprising a zeolitic material having framework type MWW, wherein the framework structure comprises Ti, Si, and O, wherein the zeolitic material further comprises Zn and an alkaline earth metal M, the molding further comprising a binder, wherein the molding exhibits a specific Lewis acidity. Further, the present invention relates to the method of preparation of said molding and the use thereof.

The present invention relates to a molding comprising a zeolitic material having framework type MWW, wherein the framework structure comprises Ti, Si, and O, wherein the zeolitic material further comprises Zn and an alkaline earth metal M, the molding further comprising a binder, wherein the molding exhibits a specific Lewis acidity.

Typically, titanium containing zeolites are used as catalysts in the on-purpose propylene oxide production by epoxidation of propylene oxide. Since usually hydrogen peroxide is used as oxidant, the industrial processes are called hydrogen peroxide to propylene oxide processes (also abbreviated herein as HPPO). In particular, two specific HPPO processes are known, whereby the one is based on a TS-1 zeolite and the other on a Zn/Ti-MWW zeolite catalyst. It has been found that the latter shows a significant improved performance over the first generation catalyst. Recent activities relate to increase the performance of the catalyst by addition of a second metal, e.g. Ba and/or La.

CN 105854933 A discloses TS-1 zeolites modified by impregnation with barium, and optionally with additional zinc and/or lanthanum. The resulting zeolites showed catalytic activity in the conversion of propylene to propylene oxide wherein hydrogen peroxide was used as oxidant and methanol as solvent.

Also, CN 106115732 A discloses TS-1 zeolites modified with barium, zinc and optionally with additional lanthanum. The prepared zeolites are shown to have catalytic activity in the liquid phase propylene epoxidation using acetonitrile as solvent.

Y. Yu et al. disclose a study on the efficiency of hydrogen peroxide utilization over titanosilicate/H₂O₂ systems. As catalysts for their study two different TS-1 zeolites, a lamellar Ti-MWW, a B-MWW, a F-Ti-MWW zeolite, a Re-Ti-MWW, and amorphous silica-alumina were prepared and tested inter alia in an epoxidation reaction of an alkene, in particular of 1-hexene.

It was an object of the present invention to provide a novel molding comprising a zeolitic material having framework type MWW, whereby the zeolitic material is particularly modified to comprise Zn and an alkaline earth metal, the molding having advantageous characteristics. In particular, it was an object to provide a novel molding having an improved propylene oxide selectivity when used as a catalyst or catalyst component, in particular in the epoxidation reaction of propene to propylene oxide. It was a further object of the present invention to provide a process for the preparation of such a molding, in particular to provide a process resulting in a molding having advantageous properties, preferably when used as a catalyst or catalyst component, specifically in an oxidation or epoxidation reaction. It was a further object of the present invention to provide an improved process for the epoxidation of propene with hydrogen peroxide as oxidizing agent, exhibiting a very low selectivity with respect to by-products and side-products of the epoxidation reaction while, at the same time, allowing for a very high propylene selectivity. Surprisingly, it was found that such a molding exhibiting said advantageous characteristics can be provided if a given molding comprising a zeolitic material having framework structure MWW is subjected to a specific subsequent water-treatment, resulting in a molding exhibiting, among others, a specific Lewis acidity determined via FTIR using pyridine as the probe gas as described herein.

Thus, it was surprisingly observed that when a precursor molding is treated in a water treatment the resulting novel molding comprising a zeolitic material having framework structure MWW showed an improved performance when used as a catalyst in the epoxidation of propene to propylene oxide by an increase of selectivity towards propylene oxide. Further, an increased lifetime of the novel molding was also observed. In particular, it has surprisingly been found that a molding can be provided which shows, if used as a catalyst in an epoxidation reaction of propene to propylene oxide and if compared to prior art moldings, significantly increased propylene oxide selectivity and yield, and further exhibits excellent life time properties.

According to the present invention, a molding is to be understood as a three-dimensional entity obtained from a shaping process; accordingly, the term “molding” is used synonymously with the term “shaped body”.

Therefore, the present invention relates to a molding, preferably the molding obtainable or obtained by the process of any one of the embodiments disclosed herein, comprising a zeolitic material having framework type MWW, having a framework structure comprising Ti, Si, and O, wherein the zeolitic material further comprises Zn and an alkaline earth metal M, the molding further comprising a binder, wherein the molding exhibits integral extinction units of the IR band at 1490 cm⁻¹ of equal to or smaller than 8. The integral extinction units of the IR band at 1490 cm⁻¹ are preferably determined as described in Reference Example 1 disclosed herein.

Further, the present invention relates to a process for preparing a molding comprising a zeolitic material having framework type MWW and a binder material, preferably the molding according to any one of the embodiments disclosed herein, the process comprising
(i) providing a molding comprising a zeolitic material having framework type MWW, having a framework structure comprising Ti, Si, and O, wherein the zeolitic material further comprises Zn, an alkaline earth metal M, and optionally a rare earth metal, wherein the molding further comprises a binder for said zeolitic material;
(ii) preparing a mixture comprising the molding according to (i) and water, and subjecting the mixture to a water treatment under hydrothermal conditions, obtaining a water-treated molding, and calcining the water-treated molding in a gas atmosphere.

Yet further, the present invention relates to a molding comprising a zeolitic material having framework type MWW and a binder material, obtainable or obtained by a process according to any one of the embodiments disclosed herein.

Yet further, the present invention relates to a use of a molding according to any one of the embodiments disclosed herein as an adsorbent, an absorbent, a catalyst or a catalyst component, preferably as a catalyst or as a catalyst component, more preferably as a Lewis acid catalyst or a Lewis acid catalyst component, as an isomerization catalyst or as an isomerization catalyst component, as an oxidation catalyst or as an oxidation catalyst component, as an aldol condensation catalyst or as an aldol condensation catalyst component, or as a Prins reaction catalyst or as a Prins reaction catalyst component, more preferably as an oxidation catalyst or as an oxidation catalyst component, more preferably as an epoxidation catalyst or as an epoxidation catalyst component, more preferably as an epoxidation catalyst.

Yet further, the present invention relates to a process for oxidizing an organic compound comprising bringing the organic compound in contact with a catalyst comprising a molding according to any one of the embodiments disclosed herein, preferably for epoxidizing an organic compound, more preferably for epoxidizing an organic compound having at least one C—C double bond, preferably a C2-C10 alkene, more preferably a C2-C5 alkene, more preferably a C2-C4 alkene, more preferably a C2 or C3 alkene, more preferably propene.

Yet further, the present invention relates to a process for preparing propylene oxide comprising reacting propene with hydrogen peroxide in acetonitrile solution in the presence of a catalyst comprising a molding according to any one of the embodiments disclosed herein to obtain propylene oxide.

With respect to the inventive molding, it is preferred that the molding integral extinction units of the IR band at 1490 cm⁻¹ in the range of from 0.05 to 8.0, more preferably in the range of from 0.1 to 7.5, more preferably in the range of from 0.5 to 7.0, more preferably in the range of from 1.0 to 6.9, more preferably in the range of from 1.5 to 6.9. It is preferred that the integral extinction units of the IR band at 1490 cm⁻¹ are determined as described in Reference Example 1 disclosed herein.

It is preferred that the molding exhibits integral extinction units of the Lewis acid IR bands in the range of from 1 to 100, more preferably in the range of from 5 to 90, more preferably in the range of from 8 to 88, more preferably in the range of from 9.0 to 79.0. It is preferred that the integral extinction units of the Lewis acid IR bands are determined as described in Reference Example 1 disclosed herein.

It is preferred that the molding exhibits integral extinction units of the Brønstedt acid IR bands of equal to or smaller than 1, preferably equal to or smaller than 0.5, more preferably equal to or smaller than 0.2, more preferably equal to or smaller than 0.1, more preferably equal to or smaller than 0.05. It is preferred that the integral extinction units of the Brønstedt acid IR bands are determined as described in Reference Example 1.

It is preferred that the molding exhibits a tortuosity parameter relative to water in the range of from 1.0 to 5.0, preferably in the range of from 1.5 to 3.0, more preferably in the range of from 1.7 to 2.5, more preferably in the range of from 1.9 to 2.1. The tortuosity parameter is preferably determined as described in Reference Example 12 disclosed herein.

According to the present invention the Brønsted acidity and the Lewis acidity were determined using an IR-spectrometer, particularly employing a FTIR-cell, wherein pyridine was used as probe gas. Preferably, a sample was pressed to a pellet. The measurement conditions preferably included heating of a sample in air to about 350° C. for about 1 h. Thus, water and any volatile substances could be removed from the sample. Further, the measurement conditions preferably included applying a low pressure (“high-vacuum” of about 10⁻⁵ mbar). Preferably, the sample cooled down to about 80° C. while applying the low pressure. The measurement was preferably conducted at about 80° C. for the entire duration of the measurement. Thus, the condensation of pyridine in the cell could be avoided. Preferably, pyridine was then dosed into the cell in successive steps (0.01, 0.1, 1, and 3 mbar). Accordingly, the controlled and complete exposition of the sample could be ensured.

It is preferred that the molding comprises Si, calculated as element, in an amount in the range of from 20 to 60 weight-%, more preferably in the range of from 30 to 55 weight-%, more preferably in the range of from 35 to 50 weight-%, more preferably in the range of from 41 to 44 weight-%, based on the total weight of the molding.

It is preferred that the molding comprises Ti, calculated as element, in an amount in the range of from 0.1 to 5 weight-%, more preferably in the range of from 0.5 to 2.0 weight-%, more preferably in the range of from 1.0 to 1.5 weight-%, based on the total weight of the molding.

It is preferred that the molding comprises Zn, calculated as element, in an amount in the range of from 0.1 to 5 weight-%, more preferably in the range of from 0.25 to 2.0 weight-%, more preferably in the range of from 0.5 to 1.0 weight-%, based on the total weight of the molding.

It is preferred that the alkaline earth metal M is one or more of Mg, Ca, Sr, and Ba, more preferably one or more of Mg, Ca, and Ba. It is particularly preferred that the alkaline earth metal M is Ba.

It is preferred that the molding comprises the alkaline earth metal M, calculated as element, in an amount in the range of from 0.1 to 5 weight-%, more preferably in the range of from 0.5 to 2.0 weight-%, more preferably in the range of from 1.0 to 1.5 weight-%, based on the total weight of the molding.

It is preferred that from 98 to 100 weight-%, preferably from 99 to 100 weight-%, more preferably from 99.5 to 100 weight-% of the molding consist of Si, O, Ti, Zn, M, and optionally H.

It is preferred that the zeolitic material further comprises a rare earth metal, more preferably one or more of Sc, Y, La, Ce, Pr, Nd, Pm, Sm, Eu, Gd, Tb, Dy, Ho, Er, Tm, Yb, and Lu, more preferably one or more of Y, La, Ce, Pr, and Nd, more preferably one or more of Y, La, and Ce, more preferably La.

In the case where the molding further comprises a rare earth metal, it is preferred that the molding comprises the rare earth metal, calculated as element, in an amount in the range of from 0.1 to 5 weight-%, more preferably in the range of from 0.25 to 2.5 weight-%, more preferably in the range of from 0.5 to 1.0 weight-%, based on the total weight of the molding.

Further in the case where the molding further comprises a rare earth metal, it is preferred that from 98 to 100 weight-%, more preferably from 99 to 100 weight-%, more preferably from 99.5 to 100 weight-% of the molding consist of Si, O, Ti, Zn, M, the rare earth metal, and optionally H.

It is preferred that the binder comprises Si and O.

It is preferred that from 95 to 100 weight-%, more preferably from 98 to 100 weight-%, more preferably from 99 to 100 weight-%, more preferably from at least 99.5 to 100 weight-%, more preferably from 99.9 to 100 weight-% of the binder comprised in the molding consist of Si and O.

It is preferred that the molding comprises the binder in an amount in the range of from 1 to 75 weight-%, more preferably in the range of from 5 to 50 weight-%, more preferably in the range of from 10 to 40 weight-%, more preferably in the range of from 15 to 25 weight-%, based on the total weight of the molding.

It is preferred that from 95 to 100 weight-%, more preferably from 98 to 100 weight-%, more preferably from 99 to 100 weight-%, more preferably from at least 99.5 to 100 weight-%, more preferably from 99.9 to 100 weight-% of the molding consist of the zeolitic material having framework type MWW and the binder.

It is preferred that the molding exhibits a total pore volume in the range of from 0.5 to 3.0 mL/g, more preferably in the range of from 0.75 to 2.5 mL/g, more preferably in the range of from 1.0 to 2.0 mL/g, more preferably in the range of from 1.25 to 1.75 mL/g. It is preferred that the pore volume is determined according to DIN 66133.

It is preferred that the molding displays a water uptake in the range of from 1 to 20 weight-%, more preferably in the range of from 6 to 15 weight-%, more preferably in the range of from 8 to 12 weight-%. It is preferred that the water uptake is determined as described in Reference Example 7.

It is preferred that the molding comprises a concentration of acid sites in the range of from 0.05 to 1.00 mmol/g, more preferably in the range of from 0.10 to 0.50 mmol/g, more preferably in the range of from 0.15 to 0.30 mmol/g, at a temperature lower than 200° C. It is preferred that the concentration of acid sites is determined by temperature programmed desorption of ammonia (NH₃-TPD) according to Reference Example 5 disclosed herein.

It is preferred that the molding comprises a concentration of acid sites of equal to or smaller than 0.05 mmol/g, more preferably of equal to or smaller than 0.02 mmol/g, at a temperature in the range of from 200 to 400° C. It is preferred that the concentration of acid sites is determined by temperature programmed desorption of ammonia (NH₃-TPD) according to Reference Example 5 disclosed herein.

It is preferred that the molding comprises a concentration of acid sites in the range of from 0.001 to 0.5 mmol/g, more preferably in the range of from 0.01 to 0.10 mmol/g, at a temperature higher than 500° C. It is preferred that the concentration of acid sites is determined by temperature programmed desorption of ammonia (NH₃-TPD) according to Reference Example 5 disclosed herein.

It is preferred that the molding is a strand, preferably having a hexagonal, rectangular, quadratic, triangular, oval, or circular cross-section, more preferably a circular cross-section.

It is preferred that the molding is a strand having a circular cross-section with a diameter in the range of from 0.5 to 5 mm, more preferably in the range of from 1 to 3 mm, more preferably in the range of from 1.5 to 2 mm.

It is preferred that the molding is an extrudate.

It is preferred that the molding, wherein the molding is preferably an extrudate, more preferably a strand as disclosed herein, exhibits a crushing strength in the range of from 5 to 50 N, more preferably in the range of from 10 to 30 N, more preferably in the range of from 15 to 25 N. It is preferred that the crushing strength is determined as described in Reference Example 6 disclosed herein.

It is preferred that the molding exhibits a propylene oxide activity of at least 6.2 weight-%, more preferably in the range of from 7.5 to 15 weight-%, more preferably in the range of from 10 to 13 weight-%. It is preferred that the propylene oxide activity is determined as described in Reference Example 8 disclosed herein.

It is preferred that the molding exhibits a propylene oxide selectivity in the range of from 96 to 100%, more preferably in the range of from 97 to 100%, more preferably in the range of from 98 to 100%. It is preferred that the propylene oxide activity is determined as described in Reference Example 9 disclosed herein.

It is preferred that the molding has a BET specific surface area equal to or greater than 100 m²/g, more preferably equal to or greater than 200 m²/g, more preferably equal to or greater than 250 m²/g, more preferably equal to or greater than 280 m²/g. It is preferred that the BET specific surface area is determined according to DIN 66131.

It is preferred that the molding is used as catalyst or catalyst component, preferably in a reaction for preparing propylene oxide from propene and hydrogen peroxide, more preferably in a reaction for continuously preparing propylene oxide from propene and hydrogen peroxide, more preferably in a continuous epoxidation reaction, more preferably in a continuous epoxidation reaction as described in Reference Example 9 disclosed herein.

Further, the present invention relates to a process for preparing a molding comprising a zeolitic material having framework type MWW and a binder material, preferably the molding according to any one of the embodiments disclosed herein, the process comprising
(i) providing a molding comprising a zeolitic material having framework type MWW, having a framework structure comprising Ti, Si, and O, wherein the zeolitic material further comprises Zn, an alkaline earth metal M, and optionally a rare earth metal, wherein the molding further comprises a binder for said zeolitic material;
(ii) preparing a mixture comprising the molding according to (i) and water, and subjecting the mixture to a water treatment under hydrothermal conditions, obtaining a water-treated molding, and calcining the water-treated molding in a gas atmosphere.

It is preferred that (i) in the process comprises
(i.1) providing a zeolitic material having framework type MWW and having a framework structure comprising Ti, Si, and O;
(i.2) providing an aqueous solution of a source of Zn;
(i.3) providing an aqueous solution of a source of an alkaline earth metal M;
(i.4) optionally providing an aqueous solution of a source of a rare earth metal;
(i.5) impregnating the zeolitic material provided according to (i.1) with the aqueous solution provided according to (i.2), the aqueous solution according to (i.3), and optionally the aqueous solution provided according to (i.4), obtaining an impregnated zeolitic material;
(i.6) preparing a mixture comprising the impregnated zeolitic material obtained from (i.5) and a binder precursor;
(i.7) shaping of the mixture obtained from (i.6).

In the case where the process comprises (i.5) as defined herein, it is preferred that (i.5) further comprises
(i.5.a) providing a mixture comprising the aqueous solution provided according to (i.2), the aqueous solution provided according to (i.3), and optionally the aqueous solution provided according to (i.4);
(i.5.b) impregnating the zeolitic material provided according to (i.1) with the mixture provided according to (i.5.a).

Alternatively, in the case where the process comprises (i.5) as defined herein, it is preferred that (i.5) comprises
(i.5.1) impregnating the zeolitic material provided according to (i.1) with the aqueous solution provided according to (i.2);
(i.5.2) impregnating the zeolitic material obtained from (i.5.1) with the aqueous solution provided according to (i.3), obtaining an impregnated zeolitic material.

Alternatively, in the case where the process comprises (i.5) as defined herein, it is preferred that
(i.5) comprises
(i.5.1′) impregnating the zeolitic material provided according to (i.1) with the aqueous solution provided according to (i.3);
(i.5.2′) impregnating the zeolitic material obtained from (i.5.1′) with the aqueous solution provided according to (i.2), obtaining an impregnated zeolitic material.

In the case where the process comprises (i.5.1) or (i.5.1′) as defined herein, it is preferred that the process further comprises
(i.5.3) optionally impregnating the zeolitic material prior to (i.5.1) or prior to (i.5.1′) with the aqueous solution provided according to (i.4);
(i.5.4) optionally impregnating the zeolitic material after (i.5.1) and prior to (i.5.2) or after
(i.5.1′) and prior to (i.5.2′) with the aqueous solution provided according to (i.4);
(i.5.5) optionally impregnating the zeolitic material after (i.5.2) or after (i.5.2′) with the aqueous solution provided according to (i.4).

Alternatively, in the case where the process comprises (i.5) as defined herein, it is preferred that
(i.5) comprises
(i.zn.1) impregnating the zeolitic material provided according to (i.1) with the aqueous solution provided according to (i.2), and optionally with the aqueous solution provided according to (i.4), obtaining an impregnated zeolitic material;
(i.zn.2) preparing a mixture comprising the impregnated zeolitic material obtained from (i.zn.1) and a binder precursor;
(i.zn.3) shaping of the mixture obtained from (i.zn.2) to obtain a first molding;
(i.zn.4) impregnating the first molding obtained from (i.zn.3) with the aqueous solution provided according to (i.3), and optionally with the aqueous solution provided according to (i.4), to obtain the precursor molding.

It is preferred that at least one of (i.5), (i.5.b), (i.5.1), (i.5.2), (i.5.1′), (i.5.2′), (i.5.3), (i.5.4), (i.5.5),
(i.zn.1), and (i.zn.4) is carried out n times, wherein n is a natural number greater than 1, wherein n preferably equal to 2, 3, 4 or 5 is.

It is preferred that the process comprises a thermal treatment in a gas atmosphere after one or more of (i.5), (i.5.b), (i.5.1), (i.5.2), (i.5.1′), (i.5.2′), (i.5.3), (i.5.4), (i.5.5), (i.zn.1), and (i.zn.4).

In the case where the process further comprises a thermal treatment after one or more of (i.5), (i.5.b), (i.5.1), (i.5.2), (i.5.1′), (i.5.2′), (i.5.3), (i.5.4), (i.5.5), (i.zn.1), and (i.zn.4), it is preferred that the thermal treatment comprises
(i.5.6) optionally drying, preferably at a temperature of the gas atmosphere in the range of from 50 to 200° C., and/or, preferably and,
(i.5.7) optionally calcining, preferably at a temperature of the gas atmosphere in the range of from 400 to 700° C.

Further in the case where the process further comprises a thermal treatment after one or more of (i.5), (i.5.b), (i.5.1), (i.5.2), (i.5.1′), (i.5.2′), (i.5.3), (i.5.4), (i.5.5), (i.zn.1), and (i.zn.4), it is preferred that the gas atmosphere comprises one or more of nitrogen, oxygen, or a mixture thereof, wherein the gas atmosphere is more preferably oxygen, air, or lean air.

It is preferred that the molding provided in (i) comprises Si, calculated as element, in an amount in the range of from 20 to 60 weight-%, more preferably in the range of from 30 to 55 weight-%, more preferably in the range of from 35 to 50 weight-%, more preferably in the range of from 40 to 45 weight-%, more preferably in the range of from 41 to 44 weight-%, based on the total weight of the molding.

It is preferred that the molding provided in (i) comprises Ti, calculated as element, in an amount in the range of from 0.01 to 10 weight-%, more preferably in the range of from 0.1 to 5 weight-%, more preferably in the range of from 0.5 to 2 weight-%, more preferably in the range of from 1.0 to 1.5 weight-%, more preferably in the range of from 1.1 to 1.4 weight-%, based on the total weight of the molding.

It is preferred that the molding provided in (i) comprises Zn, calculated as element, in an amount in the range of from 0.01 to 5 weight-%, more preferably in the range of from 0.1 to 2.5 weight-%, more preferably in the range of from 0.25 to 1.1 weight-%, more preferably in the range of from 0.5 to 0.9 weight-%, based on the total weight of the molding.

It is preferred that the alkaline earth metal M comprised in the molding provided in (i) is one or more of Mg, Ca, Sr, and Ba, more preferably one or more of Mg, Ca and Ba, wherein more preferably, the alkaline earth metal M is Ba.

It is preferred that the molding provided in (i) comprises the alkaline earth metal M, calculated as element, in an amount in the range of from 0.01 to 10 weight-%, more preferably in the range of from 0.1 to 5 weight-%, more preferably in the range of from 0.5 to 2 weight-%, more preferably in the range of from 1.0 to 1.5 weight-%, more preferably in the range of from 1.1 to 1.4 weight-%, based on the total weight of the molding.

It is preferred that the molding provided in (i) further comprises a rare earth metal, preferably one or more of Sc, Y, La, Ce, Pr, Nd, Pm, Sm, Eu, Gd, Tb, Dy, Ho, Er, Tm, Yb, and Lu, more preferably, one or more of Y, La, Ce, Pr, and Nd, more preferably one or more of Y, La, and Ce, more preferably La.

It is preferred that the molding provided in (i) further comprises a rare earth metal, preferably in an amount in the range of from 0.01 to 5 weight-%, more preferably in the range of from 0.1 to 2 weight-%, more preferably in the range of from 0.25 to 1.25 weight-%, more preferably in the range of from 0.5 to 1.0 weight-%, calculated as element and based on the total weight of the molding.

It is preferred that the molding provided in (i) comprises the binder in an amount in the range of from 1 to 50 weight-%, more preferably in the range of from 5 to 30 weight-%, more preferably in the range of from 15 to 25 weight-%, more preferably in the range of from 18 to 23 weight-%, more preferably in the range of from 19 to 22 weight-%, based on the total weight of the molding.

It is preferred that the molding provided in (i) has a bulk density in the range of from 200 to 500 g/mL, more preferably in the range of from 300 to 400 g/mL, more preferably in the range of from 325 to 375 g/mL.

It is preferred that the molding provided in (i) is a strand having a circular cross-section with a diameter in the range of from 0.5 to 5 mm, more preferably in the range of from 1 to 3 mm, more preferably in the range of from 1.5 to 2 mm, and wherein the molding exhibits a crushing strength of at least 1.5 N, preferably in the range of from 5 to 30 N, more preferably in the range of from 15 to 25 N, preferably determined as described in Reference Example 6.

It is preferred that the molding provided in (i) has a pore volume of at least 1.0 g/mL, more preferably in the range of from 1.3 to 2.0 g/mL. It is preferred that the pore volume is determined as described in Reference Example 2 disclosed herein.

It is preferred that the molding provided in (i) exhibits integral extinction units of the IR band at 1490 cm⁻¹ in the range of from 5 to 15, more preferably in the range of from 7.5 to 13.0, more preferably in the range of from 10.0 to 12.0, more preferably in the range of from 11.0 to 11.6. It is preferred that the integral extinction units of the IR band at 1490 cm⁻¹ are determined as described in Reference Example 1.

It is preferred that the molding provided in (i) exhibits integral extinction units of the Lewis acid IR bands in the range of from 1 to 100, more preferably in the range of from 50 to 200, more preferably in the range of from 75 to 150, more preferably in the range of from 101 to 125, more preferably in the range of from 105 to 120. It is preferred that the integral extinction units of the Lewis acid IR bands are determined as described in Reference Example 1.

It is preferred that the molding provided in (i) exhibits integral extinction units of the Brønstedt acid IR bands of equal to or smaller than 1, more preferably equal to or smaller than 0.5, more preferably equal to or smaller than 0.2, more preferably equal to or smaller than 0.1, more preferably equal to or smaller than 0.05. It is preferred that the Brønstedt acid IR bands are determined as described in Reference Example 1.

It is preferred that the molding provided in (i) comprises a concentration of acid sites in the range of from 0.05 to 1.00 mmol/g, more preferably in the range of from 0.10 to 0.50 mmol/g, more preferably in the range of from 0.15 to 0.25 mmol/g, at a temperature lower than 200° C. It is preferred that the concentration of acid sites is determined by temperature programmed desorption of ammonia (NH₃-TPD) according to Reference Example 5.

It is preferred that the molding provided in (i) comprises a concentration of acid sites of equal to or smaller than 0.05 mmol/g, more preferably of equal to or smaller than 0.02 mmol/g, at a temperature in the range of from 200 to 400° C. It is preferred that the concentration of acid sites is determined by temperature programmed desorption of ammonia (NH₃-TPD) according to Reference Example 5.

It is preferred that the molding provided in (i) comprises a concentration of acid sites in the range of from 0.005 to 0.1 mmol/g, more preferably in the range of from 0.01 to 0.05 mmol/g, more preferably in the range of from 0.02 to 0.03 mmol/g, at a temperature higher than 500° C. It is preferred that the concentration of acid sites is determined by temperature programmed desorption of ammonia (NH₃-TPD) according to Reference Example 5.

In the case where the process further comprises (i.1), it is preferred that the zeolitic material provided according to (i.1) comprises Si, calculated as element, in an amount in the range of from 20 to 60 weight-%, more preferably in the range of from 30 to 55 weight-%, more preferably in the range of from 35 to 50 weight-%, more preferably in the range of from 40 to 45 weight-%, more preferably in the range of from 41 to 44 weight-%, based on the total weight of the zeolitic material.

Further in the case where the process further comprises (i.1), it is preferred that the zeolitic material provided according to (i.1) comprises Ti, calculated as element, in an amount in the range of from 0.1 to 10 weight-%, more preferably in the range of from 0.5 to 5 weight-%, more preferably in the range of from 1 to 2 weight-%, more preferably in the range of from 1.2 to 1.8 weight-%, based on the total weight of the zeolitic material.

Further in the case where the process further comprises (i.1), it is preferred that the zeolitic material provided according to (i.1) comprises Zn, calculated as element, in an amount in the range of from 0.1 to 2.5 weight-%, more preferably in the range of from 0.5 to 1.3 weight-%, more preferably in the range of from 0.7 to 1.1 weight-%, based on the total weight of the molding.

Further in the case where the process further comprises (i.1), it is preferred that the alkaline earth metal M comprised in the zeolitic material provided according to (i.1) is one or more of Mg, Ca, Sr, and Ba, more preferably one or more of Mg, Ca and Ba, wherein more preferably, the alkaline earth metal M is Ba.

Further in the case where the process further comprises (i.1), it is preferred that the zeolitic material provided according to (i.1) comprises the alkaline earth metal M, calculated as element, in an amount in the range of from 0.1 to 7.5 weight-%, more preferably in the range of from 0.25 to 5 weight-%, more preferably in the range of from 0.5 to 2.5 weight-%, more preferably in the range of from 1.2 to 2.0 weight-%, based on the total weight of the molding.

Further in the case where the process further comprises (i.1), it is preferred that the zeolitic material provided according to (i.1) further comprises a rare earth metal, more preferably one or more of Sc, Y, La, Ce, Pr, Nd, Pm, Sm, Eu, Gd, Tb, Dy, Ho, Er, Tm, Yb, Lu, more preferably, one or more of Y, La, Ce, Pr, and Nd, more preferably one or more of Y, La, and Ce, more preferably La.

Further in the case where the process further comprises (i.1), it is preferred that the zeolitic material provided according to (i.1) further comprises a rare earth metal, more preferably in an amount in the range of from 0.1 to 5 weight-%, preferably in the range of from 0.25 to 2 weight-%, more preferably in the range of from 0.5 to 1.5 weight-%, more preferably in the range of from 0.8 to 1.2 weight-%, calculated as element and based on the total weight of the molding.

Further in the case where the process further comprises (i.1), it is preferred that the zeolitic material provided according to (i.1) has a crystallite size in the range of from 15 to 40 nm. It is preferred that the crystallite size is determined as described in Reference Example 4 disclosed herein.

Further in the case where the process further comprises (i.1), it is preferred that the zeolitic material provided according to (i.1) exhibits a BET specific surface area of equal to or greater than 250 m²/g, more preferably of equal to or greater than 275 m²/g, more preferably of equal to or greater than 300 m²/g. It is preferred that the BET specific surface area is determined according to DIN 66131.

Further in the case where the process further comprises (i.1), it is preferred that the zeolitic material provided according to (i.1) exhibits a C value in the range of from −150 to −40, more preferably in the range of from −125 to −50, more preferably in the range of from −100 to −60. It is preferred that the C value is determined as described in Reference Example 10 disclosed herein.

Further in the case where the process further comprises (i.1), it is preferred that the zeolitic material provided according to (i.1) exhibits a crystallinity of at least 50%, more preferably of at least 75%, more preferably of at least 80%. It is preferred that the crystallinity is determined as described in Reference Example 4 disclosed herein.

Further in the case where the process further comprises (i.1), it is preferred that the zeolitic material provided according to (i.1) has a water uptake in the range of from 8 to 20 weight-%, more preferably in the range of from 9 to 17.5 weight-%, more preferably in the range of from 10 to 15 weight-%. It is preferred that the water uptake is determined as described in Reference Example 7 disclosed herein.

Further in the case where the process further comprises (i.1), it is preferred that the zeolitic material provided according to (i.1) exhibits a propylene oxide activity of at least 10 weight-%, more preferably in the range of from 10 to 15 weight-%, more preferably in the range of from 11 to 14 weight-%. It is preferred that the propylene oxide activity is determined as described in Reference Example 8 disclosed herein.

Further in the case where the process further comprises (i.1), it is preferred that the zeolitic material provided according to (i.1) exhibits an infrared spectrum comprising a band having a maximum in the region of (3700-3750)+/−20 cm⁻¹ and a band having a maximum in the region of (3670-3690)+/−20 cm⁻¹, wherein the intensity ratio of the band in the region of (3700-3750)+/−20 cm⁻¹ relative to the band in the region of (3670-3690)+/−20 cm⁻¹ is at most 1.7, preferably at most 1.6. It is preferred that the infrared spectrum is determined as described in Reference Example 11 disclosed herein.

Further in the case where the process further comprises (i.1), it is preferred that the source of Zn is a salt, more preferably one or more of a nitrate, a halide, hydroxide, acetate, more preferably a nitrate.

Further in the case where the process further comprises (i.1), it is preferred that the alkaline earth metal in the source of the alkaline earth metal is one or more of Mg, Ca, Sr, and Ba, more preferably one or more of Mg, Ca and Ba. It is particularly preferred that the alkaline earth metal M is Ba.

Further in the case where the process further comprises (i.1), it is preferred that the source of the alkaline earth metal is a salt, more preferably one or more of a nitrate, a halide, an acetate, a hydroxide, more preferably a nitrate.

Further in the case where the process further comprises (i.2), it is preferred that the mixture according to (i.2) comprises a source of a rare earth metal, wherein the rare earth metal is one or more of Sc, Y, La, Ce, Pr, Nd, Pm, Sm, Eu, Gd, Tb, Dy, Ho, Er, Tm, Yb, and Lu, more preferably one or more of Y, La, Ce, Pr, and Nd, more preferably one or more of Y, La, and Ce, more preferably La.

In the case where the mixture according to (i.2) comprises a source of a rare earth metal, wherein the rare earth metal is one or more of Sc, Y, La, Ce, Pr, Nd, Pm, Sm, Eu, Gd, Tb, Dy, Ho, Er, Tm, Yb, and Lu, it is preferred that the source of the rare earth metal is a salt, more preferably one or more of a nitrate, a halide, and a hydroxide, more preferably a nitrate.

In the case where the process further comprises (i.5), it is preferred that impregnating according to (i.5) comprises one or more of spray-impregnation, adhesion impregnation, incipient impregnation, wet impregnation adhesion technique, and agitating, more preferably mechanically agitating, more preferably stirring, more preferably stirring for a time in the range of from 0.1 to 5 h, more preferably in the range of from 0.5 to 2 h.

Further in the case where the process further comprises (i.5), it is preferred that impregnating according to (i.5) comprises keeping the mixture at the same temperature, more preferably at a temperature in the range of from 15 to 40° C., for a time in the range of from 1 to 50 h, more preferably for a time in the range of from 30 to 40 h.

In the case where the process further comprises (i.5) and (i.6), it is preferred that after (i.5) and prior to (i.6) the process comprises
(a) optionally isolating the impregnated zeolitic material obtained in (i.5), preferably by filtration; and/or, preferably and
(b) optionally washing the impregnated zeolitic material obtained in (i.5) or (a), preferably with deionized water; and/or, preferably and
(c) optionally drying the impregnated zeolitic material obtained in (i.5), (a), or (b) in a gas atmosphere; and/or, preferably and
(d) optionally calcining the impregnated zeolitic material obtained in (i.5), (a), (b), or (c) in a gas atmosphere.

In the case where the process further comprises (c), it is preferred that drying according to (c) is carried out at a temperature of the gas atmosphere in the range of from 70 to 150° C., more preferably in the range of from 90 to 130° C., more preferably in the range of from 100 to 120° C.

Further in the case where the process further comprises (c), it is preferred that the gas atmosphere for drying in (c) comprises nitrogen, oxygen, or a mixture thereof, wherein the gas atmosphere is more preferably oxygen, air, or lean air.

Further in the case where the process further comprises (d), it is preferred that calcining according to (d) is carried out at a temperature of the gas atmosphere in the range of from 510 to 590° C., more preferably in the range of from 530 to 570° C., more preferably in the range of from 540 to 560° C.

Further in the case where the process further comprises (d), it is preferred that the gas atmosphere for calcining in (d) comprises nitrogen, oxygen, or a mixture thereof, wherein the gas atmosphere is more preferably oxygen, air, or lean air.

In the case where the process further comprises (i.6), it is preferred that the binder precursor in (i.6) is selected from the group consisting of a silica sol, a colloidal silica, a wet process silica, a dry process silica, and a mixture of two or more thereof, wherein the binder precursor is more preferably a colloidal silica.

In this context, both colloidal silica and so-called “wet process” silica and so-called “dry process” silica can be used. Colloidal silica, preferably as an alkaline and/or ammoniacal solution, more preferably as an ammoniacal solution, is commercially available, inter alia, for example as Ludox®, Syton®, Nalco® or Snowtex®. “Wet process” silica is commercially available, inter alia, for example as Hi-Sil®, Ultrasil®, Vulcasil®, Santocel®, Valron-Estersil®, Tokusil® or Nipsil®. “Dry process” silica is commercially available, inter alia, for example as Aerosil®, Reolosil®, Cab-O-Sil®, Fransil® or ArcSilica®. An ammoniacal solution of colloidal silica is preferred according to the present invention.

Further in the case where the process further comprises (i.6), it is preferred that in the mixture according to (i.6) the weight ratio of the zeolitic material obtained from (i.5) to the binder precursor is in the range of from 1:1 to 10:1, more preferably in the range of from 3:1 to 5:1, more preferably in the range of from 3.5:1 to 4.5:1.

In the case where the process further comprises (i.5) and (i.6), it is preferred that from 95 to 100 weight-%, more preferably from 98 to 100 weight-%, more preferably from 99 to 100 weight-% of the mixture prepared according to (i.6) consist of the impregnated zeolitic material obtained from (i.5), and the binder precursor.

In the case where the process further comprises (i.6), it is preferred that the mixture prepared according to (i.6) further comprises one or more viscosity modifying and/or mesopore forming agents.

In the case where the mixture prepared according to (i.6) further comprises one or more viscosity modifying and/or mesopore forming agents, it is preferred that the one or more viscosity modifying and/or mesopore forming agents are selected from the group consisting of water, alcohols, organic polymers, and mixtures of two or more thereof, wherein the organic polymers are more preferably selected from the group consisting of celluloses, cellulose derivatives, starches, polyalkylene oxides, polystyrenes, polyacrylates, polymethacrylates, polyolefins, polyamides, polyesters, and mixtures of two or more thereof, wherein the organic polymers are more preferably selected from the group consisting of cellulose derivatives, polyalkylene oxides, polystyrenes, and mixtures of two or more thereof, wherein the organic polymers are more preferably selected from the group consisting of a methyl celluloses, carboxymethylcelluloses, polyethylene oxides, polystyrenes, and mixtures of two or more thereof, wherein more preferably, the one or more viscosity modifying and/or mesopore forming agents comprise water and a methyl cellulose.

Further in the case where the mixture prepared according to (i.6) further comprises one or more viscosity modifying and/or mesopore forming agents, it is preferred that in the mixture prepared according to (i.6), the weight ratio of the zeolitic material relative to the one or more viscosity modifying and/or mesopore forming agents is in the range of from 10:1 to 20:1, more preferably in the range of from 15:1 to 16:1, more preferably in the range of from 15.5:1 to 15.7:1.

In the case where the process further comprises (i.5) and (i.6), it is preferred that from 95 to 100 weight-%, more preferably from 98 to 100 weight-%, more preferably from 99 to 100 weight-% of the mixture prepared according to (i.6) consist of the impregnated zeolitic material obtained from (i.5), the binder precursor, and the one or more viscosity modifying and/or mesopore forming agents.

In the case where the process further comprises (i.7), it is preferred that in (i.7), the mixture is shaped to a strand, more preferably to a strand having a circular cross-section.

In the case where the mixture is shaped to a strand having a circular cross-section, it is preferred that the strand having a circular cross-section has a diameter in the range of from 0.2 to 10 mm, more preferably in the range of from 0.5 to 5 mm, more preferably in the range of from 1 to 3 mm, more preferably in the range of from 1.5 to 2 mm, more preferably in the range of from 1.6 to 1.8 mm.

As regards shaping in (i.7), no particular restriction applies such that shaping may be performed by any conceivable means. In the case where the process further comprises (i.7), it is preferred that in (i.7), shaping comprises extruding the mixture.

Suitable extrusion apparatuses are described, for example, in “Ullmann's Enzyklopädie der Technischen Chemie”, 4th edition, vol. 2, page 295 et seq., 1972. In addition to the use of an extruder, an extrusion press can also be used for the preparation of the moldings. If necessary, the extruder can be suitably cooled during the extrusion process. The strands leaving the extruder via the extruder die head can be mechanically cut by a suitable wire or via a discontinuous gas stream.

In the case where the process further comprises (i.7), it is preferred that after (i.7) and prior to (ii) the process further comprises
(e) optionally drying the molding obtained from (i.7) in a gas atmosphere; and/or, preferably and
(f) optionally calcining the molding obtained from (i.7) or (e) in a gas atmosphere.

In the case where the process further comprises (e), it is preferred that drying in (e) is carried out at a temperature of the gas atmosphere in the range of from 80 to 160° C., more preferably in the range of from 100 to 140° C., more preferably in the range of from 110 to 130° C.

Further in the case where the process further comprises (e), it is preferred that the gas atmosphere for drying in (e) comprises nitrogen, oxygen, or a mixture thereof, wherein the gas atmosphere is preferably oxygen, air, or lean air.

In the case where the process further comprises (f), it is preferred that calcining according to (f) is carried out at a temperature of the gas atmosphere in the range of from 460 to 540° C., more preferably in the range of from 480 to 520° C., more preferably in the range of from 490 to 510° C.

Further in the case where the process further comprises (f), it is preferred that the gas atmosphere for calcining in (f) comprises nitrogen, oxygen, or a mixture thereof, wherein the gas atmosphere is preferably oxygen, air, or lean air.

It is preferred that the mixture in (ii) is prepared in a kneader or in a mix-muller.

It is preferred that the mixture in (ii) comprises the molding according to (i) and water in a weight ratio in the range of from 5:1 to 1:100, more preferably in the range of from 1:1 to 1:50, more preferably in the range of from 1:10 to 1:30, more preferably in the range of from 1:15 to 1:25.

It is preferred that the water-treatment according to (ii) comprises a temperature of the mixture in the range of from 100 to 200° C., more preferably in the range of from 125 to 175° C., more preferably in the range of from 130 to 160° C., more preferably in the range of from 135 to 155° C. more preferably in the range of from 140 to 150° C.

It is preferred that the water-treatment according to (ii) is carried out under autogenous pressure, more preferably in an autoclave.

It is preferred that the water-treatment according to (ii) is carried out for 6 to 10 h, more preferably for 7 to 9 h.

It is preferred that after the water-treatment and prior to the calcining in (ii), the water-treated molding is separated from the mixture obtained from the water-treatment, wherein separating preferably comprises subjecting the mixture obtained from the water-treatment to filtration or centrifugation, wherein more preferably, separating further comprises washing the water-treated molding at least once with a liquid solvent system, wherein the liquid solvent system preferably comprises one or more of water, an alcohol, and a mixture of two or more thereof, wherein the water-treated molding is more preferably washed with water.

It is preferred that, after subjecting the mixture to the water-treatment and prior to calcining the water-treated molding, (ii) further comprises drying the molding in a gas atmosphere.

In the case where the process further comprises drying, it is preferred that drying is carried out at a temperature of the gas atmosphere in the range of from 80 to 160° C., more preferably in the range of from 100 to 140° C., more preferably in the range of from 110 to 130° C.

Further I the case where the process further comprises drying, it is preferred that the gas atmosphere comprises nitrogen, oxygen, or a mixture thereof, wherein the gas atmosphere is preferably oxygen, air, or lean air.

It is preferred that calcining according to (ii) of the precursor molding, preferably of the dried precursor molding according to any one of the embodiments disclosed herein, is carried out in a gas atmosphere.

In the case where calcining according to (ii) of the precursor molding is carried out in a gas atmosphere, it is preferred that calcining is carried out at a temperature of the gas atmosphere in the range of from 410 to 490° C., more preferably in the range of from 430 to 470° C., more preferably in the range of from 440 to 460° C.

Further in the case where calcining according to (ii) of the precursor molding is carried out in a gas atmosphere, it is preferred that the gas atmosphere comprises nitrogen, oxygen, or a mixture thereof, wherein the gas atmosphere is more preferably oxygen, air, or lean air.

Yet further, the present invention relates to a molding comprising a zeolitic material having framework type MWW and a binder material, obtainable or obtained by a process according to any one of the embodiments disclosed herein.

Yet further, the present invention relates to a use of a molding according to any one of the embodiments disclosed herein as an adsorbent, an absorbent, a catalyst or a catalyst component, preferably as a catalyst or as a catalyst component, more preferably as a Lewis acid catalyst or a Lewis acid catalyst component, as an isomerization catalyst or as an isomerization catalyst component, as an oxidation catalyst or as an oxidation catalyst component, as an aldol condensation catalyst or as an aldol condensation catalyst component, or as a Prins reaction catalyst or as a Prins reaction catalyst component, more preferably as an oxidation catalyst or as an oxidation catalyst component, more preferably as an epoxidation catalyst or as an epoxidation catalyst component, more preferably as an epoxidation catalyst.

It is preferred that the molding is used for the epoxidation reaction of an organic compound having at least one C—C double bond, preferably a C2-C10 alkene, more preferably a C2-C5 alkene, more preferably a C2-C4 alkene, more preferably a C2 or C3 alkene, more preferably propene, more preferably for the epoxidation of propene with hydrogen peroxide as oxidizing agent, more preferably for the epoxidation of propene with hydrogen peroxide as oxidizing agent in a solvent comprising acetonitrile.

Yet further, the present invention relates to a process for oxidizing an organic compound comprising bringing the organic compound in contact with a catalyst comprising a molding according to any one of the embodiments disclosed herein, preferably for epoxidizing an organic compound, more preferably for epoxidizing an organic compound having at least one C—C double bond, preferably a C2-C10 alkene, more preferably a C2-C5 alkene, more preferably a C2-C4 alkene, more preferably a C2 or C3 alkene, more preferably propene.

It is preferred that hydrogen peroxide is used as oxidizing agent, wherein the oxidation reaction is more preferably carried out in a solvent, more preferably in a solvent comprising acetonitrile.

Yet further, the present invention relates to a process for preparing propylene oxide, preferably the process of any one the embodiments disclosed hereinabove, more preferably the process for oxidizing an organic compound of any one of the embodiments disclosed herein, comprising reacting propene with hydrogen peroxide in acetonitrile solution in the presence of a catalyst comprising a molding according to any one of the embodiments disclosed herein to obtain propylene oxide.

The unit bar(abs) refers to an absolute pressure of 105 Pa.

The present invention is further illustrated by the following set of embodiments and combinations of embodiments resulting from the dependencies and back-references as indicated. In particular, it is noted that in each instance where a range of embodiments is mentioned, for example in the context of a term such as “The molding of any one of embodiments 1 to 4”, every embodiment in this range is meant to be explicitly disclosed for the skilled person, i.e. the wording of this term is to be understood by the skilled person as being synonymous to “The molding of any one of embodiments 1, 2, 3, and 4”. Further, it is explicitly noted that the following set of embodiments is not the set of claims determining the extent of protection, but represents a suitably structured part of the description directed to general and preferred aspects of the present invention.
1. A molding, preferably obtainable or obtained by a process of any one of embodiments 31 to 100, comprising a zeolitic material having framework type MWW, having a framework structure comprising Ti, Si, and O, wherein the zeolitic material further comprises Zn and an alkaline earth metal M, the molding further comprising a binder, wherein the molding exhibits integral extinction units of the IR band at 1490 cm⁻¹ of equal to or smaller than 8, determined as described in Reference Example 1.
2. The molding of embodiment 1, wherein the molding exhibits integral extinction units of the IR band at 1490 cm⁻¹ in the range of from 0.05 to 8.0, preferably in the range of from 0.1 to 7.5, more preferably in the range of from 0.5 to 7.0, more preferably in the range of from 1.0 to 6.9, more preferably in the range of from 1.5 to 6.9, determined as described in Reference Example 1.
3. The molding of embodiment 1 or 2, wherein the molding exhibits integral extinction units of the Lewis acid IR bands in the range of from 1 to 100, more preferably in the range of from 5 to 90, more preferably in the range of from 8 to 88, more preferably in the range of from 9.0 to 79.0, determined as described in Reference Example 1.
4. The molding of any one of embodiments 1 to 3, wherein the molding exhibits integral extinction units of the Brønstedt acid IR bands equal to or smaller than 1, preferably equal to or smaller than 0.5, more preferably equal to or smaller than 0.2, more preferably equal to or smaller than 0.1, more preferably equal to or smaller than 0.05, determined as described in Reference Example 1.
5. The molding of any one of embodiments 1 to 4, wherein the molding exhibits a tortuosity parameter relative to water in the range of from 1.0 to 5.0, preferably in the range of from 1.5 to 3.0, more preferably in the range of from 1.7 to 2.5, preferably determined as described in Reference Example 12.
6. The molding of any one of embodiments 1 to 5, comprising Si, calculated as element, in an amount in the range of from 20 to 60 weight-%, preferably in the range of from 30 to 55 weight-%, more preferably in the range of from 35 to 50 weight-%, more preferably in the range of from 41 to 44 weight-%, based on the total weight of the molding.
7. The molding of any one of embodiments 1 to 6, comprising Ti, calculated as element, in an amount in the range of from 0.1 to 5 weight-%, preferably in the range of from 0.5 to 2.0 weight-%, more preferably in the range of from 1.0 to 1.5 weight-%, based on the total weight of the molding.
8. The molding of any one of embodiments 1 to 7, comprising Zn, calculated as element, in an amount in the range of from 0.1 to 5 weight-%, preferably in the range of from 0.25 to 2.0 weight-%, more preferably in the range of from 0.5 to 1.0 weight-%, based on the total weight of the molding.
9. The molding of any one of embodiments 1 to 8, wherein the alkaline earth metal M is one or more of Mg, Ca, Sr, and Ba, preferably one or more of Mg, Ca, and Ba, wherein more preferably, the alkaline earth metal M is Ba.
10. The molding of any one of embodiments 1 to 9, comprising the alkaline earth metal M, calculated as element, in an amount in the range of from 0.1 to 5 weight-%, preferably in the range of from 0.5 to 2.0 weight-%, more preferably in the range of from 1.0 to 1.5 weight-%, based on the total weight of the molding.
11. The molding of any one of embodiments 1 to 10, wherein from 98 to 100 weight-%, preferably from 99 to 100 weight-%, more preferably from 99.5 to 100 weight-% of the molding consist of Si, O, Ti, Zn, M, and optionally H.
12. The molding of any one of embodiments 1 to 11, wherein the zeolitic material further comprises a rare earth metal, preferably one or more of Sc, Y, La, Ce, Pr, Nd, Pm, Sm, Eu, Gd, Tb, Dy, Ho, Er, Tm, Yb, and Lu, more preferably one or more of Y, La, Ce, Pr, and Nd, more preferably one or more of Y, La, and Ce, more preferably La.
13. The molding of embodiment 12, comprising the rare earth metal, calculated as element, in an amount in the range of from 0.1 to 5 weight-%, preferably in the range of from 0.25 to 2.5 weight-%, more preferably in the range of from 0.5 to 1.0 weight-%, based on the total weight of the molding.
14. The molding of embodiment 12 or 13, wherein from 98 to 100 weight-%, preferably from 99 to 100 weight-%, more preferably from 99.5 to 100 weight-% of the molding consist of Si, O, Ti, Zn, M, the rare earth metal, and optionally H.
15. The molding of any one of embodiments 1 to 14, wherein the binder comprises Si and O.
16. The molding of any one of embodiments 1 to 15, wherein from 95 to 100 weight-%, preferably from 98 to 100 weight-%, more preferably from 99 to 100 weight-%, more preferably from at least 99.5 to 100 weight-%, more preferably from 99.9 to 100 weight-% of the binder comprised in the molding consist of Si and O.
17. The molding of any one of embodiments 1 to 16, comprising the binder in an amount in the range of from 1 to 75 weight-%, preferably in the range of from 5 to 50 weight-%, more preferably in the range of from 10 to 40 weight-%, more preferably in the range of from 15 to 25 weight-%, based on the total weight of the molding.
18. The molding of any one of embodiments 1 to 17, wherein from 95 to 100 weight-%, preferably from 98 to 100 weight-%, more preferably from 99 to 100 weight-%, more preferably from at least 99.5 to 100 weight-%, more preferably from 99.9 to 100 weight-% of the molding consist of the zeolitic material having framework type MWW and the binder.
19. The molding of any one of embodiments 1 to 18, wherein the molding exhibits a total pore volume in the range of from 0.5 to 3.0 mL/g, preferably in the range of from 0.75 to 2.5 mL/g, more preferably in the range of from 1.0 to 2.0 mL/g, more preferably in the range of from 1.25 to 1.75 mL/g, wherein the pore volume is preferably determined according to DIN 66133.
20. The molding of any one of embodiments 1 to 19, wherein the molding displays a water uptake in the range of from 1 to 20 weight-%, preferably in the range of from 6 to 15 weight-%, more preferably in the range of from 8 to 12 weight-%, wherein the water uptake is preferably determined as described in Reference Example 7.
21. The molding of any one of embodiments 1 to 20, wherein the molding comprises a concentration of acid sites in the range of from 0.05 to 1.00 mmol/g, preferably in the range of from 0.10 to 0.50 mmol/g, more preferably in the range of from 0.15 to 0.30 mmol/g, at a temperature lower than 200° C., preferably determined by temperature programmed desorption of ammonia (NH₃-TPD) according to Reference Example 5.
22. The molding of any one of embodiments 1 to 21, wherein the molding comprises a concentration of acid sites of equal to or smaller than 0.05 mmol/g, preferably of equal to or smaller than 0.02 mmol/g, at a temperature in the range of from 200 to 400° C., preferably determined by temperature programmed desorption of ammonia (NH₃-TPD) according to Reference Example 5.
23. The molding of any one of embodiments 1 to 22, wherein the molding comprises a concentration of acid sites in the range of from 0.001 to 0.5 mmol/g, preferably in the range of from 0.01 to 0.10 mmol/g, at a temperature higher than 500° C., preferably determined by temperature programmed desorption of ammonia (NH₃-TPD) according to Reference Example 5.
24. The molding of any one of embodiments 1 to 23, wherein the molding is a strand, preferably having a hexagonal, rectangular, quadratic, triangular, oval, or circular cross-section, more preferably a circular cross-section.
25. The molding of any one of embodiments 1 to 24, wherein the molding is a strand having a circular cross-section with a diameter in the range of from 0.5 to 5 mm, more preferably in the range of from 1 to 3 mm, more preferably in the range of from 1.5 to 2 mm.
26. The molding of any one of embodiments 1 to 25, wherein the molding is an extrudate.
27. The molding of any one of embodiments 1 to 26, preferably of embodiment 24 or 25, more preferably of embodiment 24, wherein the molding exhibits a crushing strength in the range of from 5 to 50 N, preferably in the range of from 10 to 30 N, more preferably in the range of from 15 to 25 N, wherein the crushing strength is preferably determined as described in Reference Example 6.
28. The molding of any one of embodiments 1 to 27, wherein the molding exhibits a propylene oxide activity of at least 6.2 weight-%, preferably in the range of from 7.5 to 15 weight-%, more preferably in the range of from 10 to 13 weight-%, preferably determined as described in Reference Example 8.
29. The molding of any one of embodiments 1 to 28, wherein the molding exhibits a propylene oxide selectivity in the range of from 96 to 100%, preferably in the range of from 97 to 100%, more preferably in the range of from 98 to 100%, preferably determined in a continuous epoxidation reaction as described in Reference Example 9.
30. The molding of any one of embodiments 1 to 29 having a BET specific surface area equal to or greater than 100 m²/g, preferably equal to or greater than 200 m²/g, more preferably equal to or greater than 250 m²/g, more preferably equal to or greater than 280 m²/g, preferably determined according to DIN 66131.
31. The molding of any one of embodiments 1 to 30, for use as catalyst or catalyst component, preferably in a reaction for preparing propylene oxide from propene and hydrogen peroxide, more preferably in a reaction for continuously preparing propylene oxide from propene and hydrogen peroxide, more preferably in a continuous epoxidation reaction as described in Reference Example 9.
32. A process for preparing a molding comprising a zeolitic material having framework type MWW and a binder material, preferably the molding according to any one of embodiments 1 to 31, the process comprising
   (i) providing a molding comprising a zeolitic material having framework type MWW, having a framework structure comprising Ti, Si, and O, wherein the zeolitic material further comprises Zn, an alkaline earth metal M, and optionally a rare earth metal, wherein the molding further comprises a binder for said zeolitic material;
   (ii) preparing a mixture comprising the molding according to (i) and water, and subjecting the mixture to a water treatment under hydrothermal conditions, obtaining a water-treated molding, and calcining the water-treated molding in a gas atmosphere.
33. The process of embodiment 32, wherein (i) comprises
   (i.1) providing a zeolitic material having framework type MWW and having a framework structure comprising Ti, Si, and O;
   (i.2) providing an aqueous solution of a source of Zn;
   (i.3) providing an aqueous solution of a source of an alkaline earth metal M;
   (i.4) optionally providing an aqueous solution of a source of a rare earth metal;
   (i.5) impregnating the zeolitic material provided according to (i.1) with the aqueous solution provided according to (i.2), the aqueous solution according to (i.3), and optionally the aqueous solution provided according to (i.4), obtaining an impregnated zeolitic material;
   (i.6) preparing a mixture comprising the impregnated zeolitic material obtained from (i.5) and a binder precursor;
   (i.7) shaping of the mixture obtained from (i.6).
34. The process of embodiment 33, wherein (i.5) comprises
   (i.5.a) providing a mixture comprising the aqueous solution provided according to (i.2), the aqueous solution provided according to (i.3), and optionally the aqueous solution provided according to (i.4);
   (i.5.b) impregnating the zeolitic material provided according to (i.1) with the mixture provided according to (i.5.a).
35. The process of any one of embodiments 32 to 34, wherein the molding provided in (i) comprises Si, calculated as element, in an amount in the range of from 20 to 60 weight-%, preferably in the range of from 30 to 55 weight-%, more preferably in the range of from 35 to 50 weight-%, more preferably in the range of from 40 to 45 weight-%, more preferably in the range of from 41 to 44 weight-%, based on the total weight of the molding.
36. The process of any one of embodiments 32 to 35, wherein the molding provided in (i) comprises Ti, calculated as element, in an amount in the range of from 0.01 to 10 weight-%, preferably in the range of from 0.1 to 5 weight-%, more preferably in the range of from 0.5 to 2 weight-%, more preferably in the range of from 1.0 to 1.5 weight-%, more preferably in the range of from 1.1 to 1.4 weight-%, based on the total weight of the molding.
37. The process of any one of embodiments 32 to 36, wherein the molding provided in (i) comprises Zn, calculated as element, in an amount in the range of from 0.01 to 5 weight-%, preferably in the range of from 0.1 to 2.5 weight-%, more preferably in the range of from 0.25 to 1.1 weight-%, more preferably in the range of from 0.5 to 0.9 weight-%, based on the total weight of the molding.
38. The process of any one of embodiments 32 to 37, wherein the alkaline earth metal M comprised in the molding provided in (i) is one or more of Mg, Ca, Sr, and Ba, preferably one or more of Mg, Ca and Ba, wherein more preferably, the alkaline earth metal M is Ba.
39. The process of any one of embodiments 32 to 38, wherein the molding provided in (i) comprises the alkaline earth metal M, calculated as element, in an amount in the range of from 0.01 to 10 weight-%, preferably in the range of from 0.1 to 5 weight-%, more preferably in the range of from 0.5 to 2 weight-%, more preferably in the range of from 1.0 to 1.5 weight-%, more preferably in the range of from 1.1 to 1.4 weight-%, based on the total weight of the molding.
40. The process of any one of embodiments 32 to 39, wherein the molding provided in (i) further comprises a rare earth metal, preferably one or more of Sc, Y, La, Ce, Pr, Nd, Pm, Sm, Eu, Gd, Tb, Dy, Ho, Er, Tm, Yb, and Lu, more preferably, one or more of Y, La, Ce, Pr, and Nd, more preferably one or more of Y, La, and Ce, more preferably La.
41. The process of any one of embodiments 32 to 40, wherein the molding provided in (i) further comprises a rare earth metal, preferably in an amount in the range of from 0.01 to 5 weight-%, more preferably in the range of from 0.1 to 2 weight-%, more preferably in the range of from 0.25 to 1.25 weight-%, more preferably in the range of from 0.5 to 1.0 weight-%, calculated as element and based on the total weight of the molding.
42. The process of any one of embodiments 32 to 41, wherein the molding provided in (i) comprises the binder in an amount in the range of from 1 to 50 weight-%, preferably in the range of from 5 to 30 weight-%, more preferably in the range of from 15 to 25 weight-%, more preferably in the range of from 18 to 23 weight-%, more preferably in the range of from 19 to 2221 weight-%, based on the total weight of the molding.
43. The process of any one of embodiments 32 to 42, wherein the molding provided in (i) has a bulk density in the range of from 200 to 500 g/mL, preferably in the range of from 300 to 400 g/mL, more preferably in the range of from 325 to 375 g/mL.
44. The process of any one of embodiments 32 to 43, wherein the molding provided in (i) is a strand having a circular cross-section with a diameter in the range of from 0.5 to 5 mm, preferably in the range of from 1 to 3 mm, more preferably in the range of from 1.5 to 2 mm, and wherein the molding exhibits a crushing strength of at least 1.5 N, preferably in the range of from 5 to 30 N, more preferably in the range of from 15 to 25 N, preferably determined as described in Reference Example 6.
45. The process of any one of embodiments 32 to 44, wherein the molding provided in (i) has a pore volume of at least 1.0 g/mL, preferably in the range of from 1.3 to 2.0 g/mL, preferably determined as described in Reference Example 2.
46. The process of any one of embodiments 32 to 45 wherein the molding provided in (i) exhibits integral extinction units of the IR band at 1490 cm¹ in the range of from 5 to 15, more preferably in the range of from 7.5 to 13.0, more preferably in the range of from 10.0 to 12.0, more preferably in the range of from 11.0 to 11.6, determined as described in Reference Example 1.
47. The process of any one of embodiments 32 to 46, wherein the molding provided in (i) exhibits integral extinction units of the Lewis acid IR bands in the range of from 50 to 200, more preferably in the range of from 75 to 150, more preferably in the range of from 101 to 125, more preferably in the range of from 105 to 120, determined as described in Reference Example 1.
48. The process of any one of embodiments 32 to 47, wherein the molding provided in (i) exhibits integral extinction units of the Brønstedt acid IR bands of equal to or smaller than 1, preferably equal to or smaller than 0.5, more preferably equal to or smaller than 0.2, more preferably equal to or smaller than 0.1, more preferably equal to or smaller than 0.01, determined as described in Reference Example 1.
49. The process of any one of embodiments 32 to 48, wherein the molding provided in (i) comprises a concentration of acid sites in the range of from 0.05 to 1.00 mmol/g, preferably in the range of from 0.10 to 0.50 mmol/g, at a temperature lower than 200° C., preferably determined by temperature programmed desorption of ammonia (NH₃-TPD) according to Reference Example 5.
50. The process of any one of embodiments 32 to 49, wherein the molding provided in (i) comprises a concentration of acid sites of at most 0.05 mmol/g, preferably of at most 0.02 mmol/g, at a temperature in the range of from 200 to 400° C., preferably determined by temperature programmed desorption of ammonia (NH₃-TPD) according to Reference Example 5.
51. The process of any one of embodiments 32 to 50, wherein the molding provided in (i) comprises a concentration of acid sites in the range of from 0.005 to 0.1 mmol/g, preferably in the range of from 0.01 to 0.05 mmol/g, more preferably in the range of from 0.02 to 0.03 mmol/g, at a temperature higher than 500° C., preferably determined by temperature programmed desorption of ammonia (NH₃-TPD) according to Reference Example 5.
52. The process of any one of embodiments 33 to 51, wherein the zeolitic material provided according to (i.1) comprises Si, calculated as element, in an amount in the range of from 20 to 60 weight-%, preferably in the range of from 30 to 55 weight-%, more preferably in the range of from 35 to 50 weight-%, more preferably in the range of from 40 to 45 weight-%, more preferably in the range of from 41 to 44 weight-%, based on the total weight of the zeolitic material.
53. The process of any one of embodiments 33 to 52, wherein the zeolitic material provided according to (i.1) comprises Ti, calculated as element, in an amount in the range of from 0.1 to 10 weight-%, preferably in the range of from 0.5 to 5 weight-%, more preferably in the range of from 1 to 2 weight-%, more preferably in the range of from 1.2 to 1.8 weight-%, based on the total weight of the zeolitic material.
54. The process of any one of embodiments 33 to 53, wherein the zeolitic material provided according to (i.1) comprises Zn, calculated as element, in an amount in the range of from 0.1 to 2.5 weight-%, preferably in the range of from 0.5 to 1.3 weight-%, more preferably in the range of from 0.7 to 1.1 weight-%, based on the total weight of the molding.
55. The process of any one of embodiments 33 to 54, wherein the alkaline earth metal M comprised in the zeolitic material provided according to (i.1) is one or more of Mg, Ca, Sr, and Ba, preferably one or more of Mg, Ca and Ba, wherein more preferably, the alkaline earth metal M is Ba.
56. The process of any one of embodiments 33 to 55, wherein the zeolitic material provided according to (i.1) comprises the alkaline earth metal M, calculated as element, in an amount in the range of from 0.1 to 7.5 weight-%, preferably in the range of from 0.25 to 5 weight-%, more preferably in the range of from 0.5 to 2.5 weight-%, more preferably in the range of from 1.2 to 2.0 weight-%, based on the total weight of the molding.
57. The process of any one of embodiments 33 to 56, wherein the zeolitic material provided according to (i.1) further comprises a rare earth metal, preferably one or more of Sc, Y, La, Ce, Pr, Nd, Pm, Sm, Eu, Gd, Tb, Dy, Ho, Er, Tm, Yb, Lu, more preferably, one or more of Y, La, Ce, Pr, and Nd, more preferably one or more of Y, La, and Ce, more preferably La.
58. The process of any one of embodiments 33 to 57, wherein the zeolitic material provided according to (i.1) further comprises a rare earth metal, preferably in an amount in the range of from 0.1 to 5 weight-%, preferably in the range of from 0.25 to 2 weight-%, more preferably in the range of from 0.5 to 1.5 weight-%, more preferably in the range of from 0.8 to 1.2 weight-%, calculated as element and based on the total weight of the molding.
59. The process of any one of embodiments 33 to 58, wherein the zeolitic material provided according to (i.1) has a crystallite size in the range of from 15 to 40 nm, preferably determined described in Reference Example 4.
60. The process of any one of embodiments 33 to 59, wherein the zeolitic material provided according to (i.1) exhibits a BET specific surface area of equal to or greater than 250 m²/g, preferably of equal to or greater than 275 m²/g, more preferably of equal to or greater than 300 m²/g, preferably determined according to DIN 66131.
61. The process of any one of embodiments 33 to 60, wherein the zeolitic material provided according to (i.1) exhibits a C value in the range of from −150 to −40, preferably in the range of from −125 to −50, more preferably in the range of from −100 to −60, preferably determined as described in Reference Example 10.
62. The process of any one of embodiments 33 to 61, wherein the zeolitic material provided according to (i.1) exhibits a crystallinity of at least 50%, preferably of at least 75%, more preferably of at least 80%, preferably determined as described in Reference Example 4.
63. The process of any one of embodiments 33 to 62, wherein the zeolitic material provided according to (i.1) has a water uptake in the range of from 8 to 20 weight-%, preferably in the range of from 9 to 17.5 weight-%, more preferably in the range of from 10 to 15 weight-%, preferably determined as described in Reference Example 7.
64. The process of any one of embodiments 33 to 63, wherein the zeolitic material provided according to (i.1) exhibits a propylene oxide activity of at least 10 weight-%, preferably in the range of from 10 to 15 weight-%, more preferably in the range of from 11 to 14 weight-%, preferably determined as described in Reference Example 8.
65. The process of any one of embodiments 33 to 64, wherein the zeolitic material provided according to (i.1) exhibits an infrared spectrum comprising a band having a maximum in the region of (3700−3750)+/−20 cm⁻¹ and a band having a maximum in the region of (3670-3690)+/−20 cm⁻¹, wherein the intensity ratio of the band in the region of (3700 3750)+/−20 cm⁻¹ relative to the band in the region of (3670-3690)+/−20 cm⁻¹ is at most 1.7, preferably at most 1.6, preferably determined as described in Reference Example 11.
66. The process of any one of embodiments 33 to 65, wherein the source of Zn is a salt, preferably one or more of a nitrate, a halide, hydroxide, acetate, preferably a nitrate.
67. The process of any one of embodiments 33 to 66, wherein the alkaline earth metal in the source of the alkaline earth metal is one or more of Mg, Ca, Sr, and Ba, preferably one or more of Mg, Ca and Ba, wherein more preferably, the alkaline earth metal M is Ba.
68. The process of any one of embodiments 33 to 67, wherein the source of the alkaline earth metal is a salt, preferably one or more of a nitrate, a halide, an acetate, a hydroxide, more preferably a nitrate.
69. The process of any one of embodiments 33 to 68, wherein the mixture according to (i.2) comprises a source of a rare earth metal, wherein the rare earth metal is one or more of Sc, Y, La, Ce, Pr, Nd, Pm, Sm, Eu, Gd, Tb, Dy, Ho, Er, Tm, Yb, and Lu, more preferably one or more of Y, La, Ce, Pr, and Nd, more preferably one or more of Y, La, and Ce, more preferably La.
70. The process of embodiment 69, wherein the source of the rare earth metal is a salt, preferably one or more of a nitrate, a halide, and a hydroxide, more preferably a nitrate.
71. The process of any one of embodiments 33 to 70, wherein impregnating according to (i.5) comprises one or more of spray-impregnation, adhesion impregnation, incipient impregnation, wet impregnation adhesion technique, and agitating, preferably mechanically agitating, more preferably stirring, more preferably stirring for a time in the range of from 0.1 to 5 h, more preferably in the range of from 0.5 to 2 h.
72. The process of any one of embodiments 33 to 71, wherein impregnating according to (i.5) comprises keeping the mixture at the same temperature, preferably at a temperature in the range of from 15 to 40° C., for a time in the range of from 1 to 50 h, preferably for a time in the range of from 30 to 40 h.
73. The process of any one of embodiments 33 to 72, wherein after (i.5) and prior to (i.6) the process comprises
   (a) optionally isolating the impregnated zeolitic material obtained in (i.5), preferably by filtration; and/or, preferably and
   (b) optionally washing the impregnated zeolitic material obtained in (i.5) or (a), preferably with deionized water; and/or, preferably and
   (c) optionally drying the impregnated zeolitic material obtained in (i.5), (a), or (b) in a gas atmosphere; and/or, preferably and
   (d) optionally calcining the impregnated zeolitic material obtained in (i.5), (a), (b), or (c) in a gas atmosphere.
74. The process of embodiment 73, wherein drying according to (c) is carried out at a temperature of the gas atmosphere in the range of from 70 to 150° C., preferably in the range of from 90 to 130° C., more preferably in the range of from 100 to 120° C.
75. The process of embodiment 73 or 74, wherein the gas atmosphere for drying in (c) comprises nitrogen, oxygen, or a mixture thereof, wherein the gas atmosphere is preferably oxygen, air, or lean air.
76. The process of any one of embodiments 73 to 75, wherein calcining according to (d) is carried out at a temperature of the gas atmosphere in the range of from 510 to 590° C., preferably in the range of from 530 to 570° C., more preferably in the range of from 540 to 560° C.
77. The process of embodiment 73 or 76, wherein the gas atmosphere for calcining in (d) comprises nitrogen, oxygen, or a mixture thereof, wherein the gas atmosphere is preferably oxygen, air, or lean air.
78. The process of any one of embodiments 33 to 77, wherein the binder precursor is selected from the group consisting of a silica sol, a colloidal silica, a wet process silica, a dry process silica, and a mixture of two or more thereof, wherein the binder precursor is more preferably a colloidal silica.
79. The process of any one of embodiments 33 to 78, wherein in the mixture according to (i.6) the weight ratio of the zeolitic material obtained from (i.5) to the binder precursor is in the range of from 1:1 to 10:1, preferably in the range of from 3:1 to 5:1, more preferably in the range of from 3.5:1 to 4.5:1.
80. The process of any one of embodiments 33 to 79, wherein from 95 to 100 weight-%, preferably from 98 to 100 weight-%, more preferably from 99 to 100 weight-% of the mixture prepared according to (i.6) consist of the impregnated zeolitic material obtained from (i.5), and the binder precursor.
81. The process of any one of embodiments 33 to 80, wherein the mixture prepared according to (i.6) further comprises one or more viscosity modifying and/or mesopore forming agents.
82. The process of embodiment 81, wherein the one or more viscosity modifying and/or mesopore forming agents are selected from the group consisting of water, alcohols, organic polymers, and mixtures of two or more thereof, wherein the organic polymers are preferably selected from the group consisting of celluloses, cellulose derivatives, starches, polyalkylene oxides, polystyrenes, polyacrylates, polymethacrylates, polyolefins, polyamides, polyesters, and mixtures of two or more thereof, wherein the organic polymers are more preferably selected from the group consisting of cellulose derivatives, polyalkylene oxides, polystyrenes, and mixtures of two or more thereof, wherein the organic polymers are more preferably selected from the group consisting of a methyl celluloses, carboxymethylcelluloses, polyethylene oxides, polystyrenes, and mixtures of two or more thereof, wherein more preferably, the one or more viscosity modifying and/or mesopore forming agents comprise water and a methyl cellulose.
83. The process of embodiment 81 or 82, wherein in the mixture prepared according to (i.6), the weight ratio of the zeolitic material, relative to the one or more viscosity modifying and/or mesopore forming agents is in the range of from 10:1 to 20:1, preferably in the range of from 15:1 to 16:1, more preferably in the range of from 15.5:1 to 15.7:1.
84. The process of any one of embodiments 33 to 83, wherein from 95 to 100 weight-%, preferably from 98 to 100 weight-%, more preferably from 99 to 100 weight-% of the mixture prepared according to (i.6) consist of the impregnated zeolitic material obtained from (i.5), the binder precursor, and the one or more viscosity modifying and/or mesopore forming agents.
85. The process of any one of embodiments 33 to 84, wherein in (i.7), the mixture is shaped to a strand, preferably to a strand having a circular cross-section.
86. The process of embodiment 85, wherein the strand having a circular cross-section has a diameter in the range of from 0.2 to 10 mm, preferably in the range of from 0.5 to 5 mm, more preferably in the range of from 1 to 3 mm, more preferably in the range of from 1.5 to 2 mm, more preferably in the range of from 1.6 to 1.8 mm.
87. The process of any one of embodiments 33 to 86, wherein in (i.7), shaping comprises extruding the mixture.
88. The process of any one of embodiments 33 to 87, wherein after (i.7) and prior to (ii) the process further comprises
   (e) optionally drying the molding obtained from (i.7) in a gas atmosphere; and/or, preferably and
   (f) optionally calcining the molding obtained from (i.7) or (e) in a gas atmosphere.
89. The process of embodiment 88, wherein drying in (e) is carried out at a temperature of the gas atmosphere in the range of from 80 to 160° C., preferably in the range of from 100 to 140° C., more preferably in the range of from 110 to 130° C.
90. The process of embodiment 88 or 89, wherein the gas atmosphere for drying in (e) comprises nitrogen, oxygen, or a mixture thereof, wherein the gas atmosphere is preferably oxygen, air, or lean air.
91. The process of any one of embodiments 88 to 90, wherein calcining according to (f) is carried out at a temperature of the gas atmosphere in the range of from 460 to 540° C., preferably in the range of from 480 to 520° C., more preferably in the range of from 490 to 510° C.
92. The process of any one of embodiments 88 to 91, wherein the gas atmosphere for calcining in (f) comprises nitrogen, oxygen, or a mixture thereof, wherein the gas atmosphere is preferably oxygen, air, or lean air.
93. The process of any one of embodiments 32 to 92, wherein the mixture in (ii) is prepared in a kneader or in a mix-muller.
94. The process of any one of embodiments 32 to 93, wherein the mixture in (ii) comprises the molding according to (i) and water in a weight ratio in the range of from 5:1 to 1:100, preferably in the range of from 1:1 to 1:50, more preferably in the range of from 1:10 to 1:30, more preferably in the range of from 1:15 to 1:25.
95. The process of any one of embodiments 32 to 94, wherein the water-treatment according to (ii) comprises a temperature of the mixture in the range of from 100 to 200° C., preferably in the range of from 125 to 175° C., more preferably in the range of from 130 to 160° C., more preferably in the range of from 135 to 155° C. more preferably in the range of from 140 to 150° C.
96. The process of any one of embodiments 32 to 95, wherein the water-treatment according to (ii) is carried out under autogenous pressure, preferably in an autoclave.
97. The process of any one of embodiments 32 to 96, wherein the water-treatment according to (ii) is carried out for 6 to 10 h, preferably for 7 to 9 h.
98. The process of any one of embodiments 32 to 97, wherein after the water-treatment and prior to the calcining in (ii), the water-treated molding is separated from the mixture obtained from the water-treatment, wherein separating preferably comprises subjecting the mixture obtained from the water-treatment to filtration or centrifugation, wherein more preferably, separating further comprises washing the water-treated molding at least once with a liquid solvent system, wherein the liquid solvent system preferably comprises one or more of water, an alcohol, and a mixture of two or more thereof, wherein the water-treated molding is more preferably washed with water.
99. The process of any one of embodiments 32 to 98, wherein after subjecting the mixture to the water-treatment and prior to calcining the water-treated molding (ii) further comprises drying the molding in a gas atmosphere.
100. The process of embodiment 99, wherein drying is carried out at a temperature of the gas atmosphere in the range of from 80 to 160° C., preferably in the range of from 100 to 140° C., more preferably in the range of from 110 to 130° C.
101. The process of embodiment 99 or 100, wherein the gas atmosphere comprises nitrogen, oxygen, or a mixture thereof, wherein the gas atmosphere is preferably oxygen, air, or lean air.
102. The process of any one of embodiments 32 to 101, preferably of any one of embodiments 93 to 101, wherein calcining according to (ii) of the molding, preferably of the dried molding according to any one of embodiments 86 to 90, is carried out in a gas atmosphere.
103. The process of embodiment 102, wherein calcining is carried out at a temperature of the gas atmosphere in the range of from 410 to 490° C., preferably in the range of from 430 to 470° C., more preferably in the range of from 440 to 460° C.
104. The process of embodiment 102 or 103, wherein the gas atmosphere comprises nitrogen, oxygen, or a mixture thereof, wherein the gas atmosphere is preferably oxygen, air, or lean air.
105. A molding comprising a zeolitic material having framework type MWW and a binder material, obtainable or obtained by a process according to any one of embodiments 32 to 104.
106. Use of a molding according to any one of embodiments 1 to 31 or according to embodiment 105 as an adsorbent, an absorbent, a catalyst or a catalyst component, preferably as a catalyst or as a catalyst component, more preferably as a Lewis acid catalyst or a Lewis acid catalyst component, as an isomerization catalyst or as an isomerization catalyst component, as an oxidation catalyst or as an oxidation catalyst component, as an aldol condensation catalyst or as an aldol condensation catalyst component, or as a Prins reaction catalyst or as a Prins reaction catalyst component, more preferably as an oxidation catalyst or as an oxidation catalyst component, more preferably as an epoxidation catalyst or as an epoxidation catalyst component, more preferably as an epoxidation catalyst.
107. The use of embodiment 106 for the epoxidation reaction of an organic compound having at least one C—C double bond, preferably a C2-C10 alkene, more preferably a C2-C5 alkene, more preferably a C2-C4 alkene, more preferably a C2 or C3 alkene, more preferably propene, more preferably for the epoxidation of propene with hydrogen peroxide as oxidizing agent, more preferably for the epoxidation of propene with hydrogen peroxide as oxidizing agent in a solvent comprising acetonitrile.
108. A process for oxidizing an organic compound comprising bringing the organic compound in contact with a catalyst comprising a molding according to any one of embodiments 1 to 31 or according to embodiment 105, preferably for epoxidizing an organic compound, more preferably for epoxidizing an organic compound having at least one C—C double bond, preferably a C2-C10 alkene, more preferably a C2-C5 alkene, more preferably a C2-C4 alkene, more preferably a C2 or C3 alkene, more preferably propene.
109. The process of embodiment 108, wherein hydrogen peroxide is used as oxidizing agent, wherein the oxidation reaction is preferably carried out in a solvent, more preferably in a solvent comprising acetonitrile.
110. A process, preferably the process of embodiment 108 or 109, for preparing propylene oxide comprising reacting propene with hydrogen peroxide in acetonitrile solution in the presence of a catalyst comprising a molding according to any one of embodiments 1 to 31 or according to embodiment 105 to obtain propylene oxide.

The present invention is further illustrated by the following examples and reference examples.

### REFERENCE EXAMPLE 1: DETERMINATION OF BRøNSTEDT AND LEWIS ACIDITY

In the examples, the Brønsted and Lewis acidities were determined using pyridine as the probe gas. The measurements were conducted using an IR-spectrometer Nicolet 6700 employing a FTIR-cell. The samples were pressed to a pellet for placing in the FTIR-cell for measurement. After being placed in the FTIR-cell, the samples were then heated in air to 350° C. and held at that temperature for 1 h for removing water and any volatile substances from the sample. The apparatus was then placed under high-vacuum (10-5 mbar), and the cell let cool to 80° C., at which it was held for the entire duration of the measurement for avoiding the condensation of pyridine in the cell.

Pyridine was then dosed into the cell in successive steps (0.01, 0.1, 1, and 3 mbar) to ensure the controlled and complete exposition of the sample.

The irradiation spectrum of the activated sample at 80° C. and 10-5 mbar was used as the background for the absorption spectra for compensating the influence of matrix bands.

For the analysis, the spectrum at a pressure of 1 mbar was used, since the sample was in a stable equilibrium. For the quantification, the extinction spectrum was used, since this allowed for the cancellation of the matrix effects.

The integral extinction units were determined as follows: the characteristic signals for the pyridine absorption were integrated and the thus determined area was scaled with the thickness of the pellet. For allowing a better comparison, the determined values were multiplied with a constant factor, said factor being 1000. Accordingly, the integral extinction units were calculated based on the measured spectrum according to formula I:

Integral extinction units=(Area below an extinction band at 1 mbar/value of thickness of disassembled pellet in μm)×1000.

The integral extinction units (integrale Extinktionseinheiten) of the IR bands at a pressure of 1 mbar are used herein as a value to define the Lewis acidity of a respective material. Further, the integral extinction units of the IR band at 1490 cm⁻¹ at a pressure of 1 mbar are used herein as a further value to define the acidity of a respective material.

TABLE 1
Assignment of the IR-bands of pyridine

| acid sites | pyridine species | bands (cm⁻¹) |
|---|---|---|
| L | Py | 1440-1455 |
| | | 1575 |
| | | 1620 |
| B | PyH⁺ | 1540-1550 |
| | | 1635-1640 |
| B + L | Py + PyH⁺ | 1490 |
| physical adsorbate | adsorbated Py | 1440 (overlay L) |
| | | 1580-1595 |

Py = pyridine;
PyH⁺ = pyridinium ion;
B = Brønsted center;
L = Lewis center

In the examples, the determination of the Lewis acid sites were determined considering the band at 1450 cm⁻¹ and of the Brønsted acid sites considering the band at 1545 cm⁻¹.

### REFERENCE EXAMPLE 2: DETERMINATION OF THE TOTAL PORE VOLUME

The total pore volume was determined via intrusion mercury porosimetry according to DIN 66133.

### REFERENCE EXAMPLE 3: DETERMINATION OF THE BET SPECIFIC SURFACE AREA

The BET specific surface area was determined via nitrogen physisorption at 77 K according to the method disclosed in DIN 66131. The N₂ sorption isotherms at the temperature of liquid nitrogen were measured using Micrometrics ASAP 2020M and Tristar system for determining the BET specific surface area.

### REFERENCE EXAMPLE 4: X-RAY POWDER DIFFRACTION AND DETERMINATION OF THE CRYSTALLINITY

Powder X-ray diffraction (PXRD) data was collected using a diffractometer (D8 Advance Series II, Bruker AXS GmbH) equipped with a LYNXEYE detector operated with a Copper anode X-ray tube running at 40 kV and 40 mA. The geometry was Bragg-Brentano, and air scattering was reduced using an air scatter shield.

Computing crystallinity: The crystallinity of the samples was determined using the software DIFFRAC.EVA provided by Bruker AXS GmbH, Karlsruhe. The method is described on page 121 of the user manual. The default parameters for the calculation were used.

Computing phase composition: The phase composition was computed against the raw data using the modelling software DIFFRAC.TOPAS provided by Bruker AXS GmbH, Karlsruhe. The crystal structures of the identified phases, instrumental parameters as well the crystallite size of the individual phases were used to simulate the diffraction pattern. This was fit against the data in addition to a function modelling the background intensities.

Data collection: The samples were homogenized in a mortar and then pressed into a standard flat sample holder provided by Bruker AXS GmbH for Bragg-Brentano geometry data collection. The flat surface was achieved using a glass plate to compress and flatten the sample powder. The data was collected from the angular range 2 to 70 ° 2Theta with a step size of 0.02° 2Theta, while the variable divergence slit was set to an angle of 0.1°. The crystalline content describes the intensity of the crystalline signal to the total scattered intensity. (User Manual for DIFFRAC.EVA, Bruker AXS GmbH, Karlsruhe.)

### REFERENCE EXAMPLE 5: DETERMINATION OF THE ACID SITES: TEMPERATURE PROGRAMMED DESORPTION OF AMMONIA (NH₃-TPD)

The temperature-programmed desorption of ammonia (NH₃-TPD) was conducted in an automated chemisorption analysis unit (Micromeritics AutoChem II 2920) having a thermal conductivity detector. Continuous analysis of the desorbed species was accomplished using an online mass spectrometer (OmniStar QMG200 from Pfeiffer Vacuum). The sample (0.1 g) was introduced into a quartz tube and analysed using the program described below. The temperature was measured by means of a Ni/Cr/Ni thermocouple immediately above the sample in the quartz tube. For the analyses, He of purity 5.0 was used. Before any measurement, a blank sample was analysed for calibration.
1. Preparation: Commencement of recording; one measurement per second. Wait for 10 minutes at 25° C. and a He flow rate of 30 cm³/min (room temperature (about 25° C.) and 1 atm); heat up to 600° C. at a heating rate of 20 K/min; hold for 10 minutes. Cool down under a He flow (30 cm³/min) to 100° C. at a cooling rate of 20 K/min (furnace ramp temperature); Cool down under a He flow (30 cm³/min) to 100° C. at a cooling rate of 3 K/min (sample ramp temperature).
2. Saturation with NH₃: Commencement of recording; one measurement per second. Change the gas flow to a mixture of 10% NH₃ in He (75 cm³/min; 100° C. and 1 atm) at 100° C.; hold for 30 min.
3. Removal of the excess: Commencement of recording; one measurement per second. Change the gas flow to a He flow of 75 cm³/min (100° C. and 1 atm) at 100° C.; hold for 60 min.
4. NH₃-TPD: Commencement of recording; one measurement per second. Heat up under a He flow (flow rate: 30 cm³/min) to 600° C. at a heating rate of 10 K/min; hold for 30 min.
5. End of measurement.

Desorbed ammonia was measured by means of the online mass spectrometer, which demonstrated that the signal from the thermal conductivity detector was caused by desorbed ammonia. This involved utilizing the m/z=16 signal from ammonia in order to monitor the desorption of the ammonia. The amount of ammonia adsorbed (mmol/g of sample) was ascertained by means of the Micromeritics software through integration of the TPD signal with a horizontal baseline.

### REFERENCE EXAMPLE 6: DETERMINATION OF THE HARDNESS

The crush strength as referred to in the context of the present invention is to be understood as having been determined via a crush strength test machine Z2.5/TS1S, supplier Zwick GmbH & Co., D-89079 Ulm, Germany. As to fundamentals of this machine and its operation, reference is made to the respective instructions handbook “Register 1: Betriebsanleitung/Sicherheitshandbuch für die Material-Prüfmaschine Z2.5/TS1S”, version 1.5, December 2001 by Zwick GmbH & Co. Technische Dokumentation, August-Nagel-Strasse 11, D-89079 Ulm, Germany. The machine was equipped with a fixed horizontal table on which the strand was positioned. A plunger having a diameter of 3 mm which was freely movable in vertical direction actuated the strand against the fixed table. The apparatus was operated with a preliminary force of 0.5 N, a shear rate under preliminary force of 10 mm/min and a subsequent testing rate of 1.6 mm/min. The vertically movable plunger was connected to a load cell for force pick-up and, during the measurement, moved toward the fixed turntable on which the molding (strand) to be investigated is positioned, thus actuating the strand against the table. The plunger was applied to the strands perpendicularly to their longitudinal axis. With said machine, a given strand as described below was subjected to an increasing force via a plunger until the strand was crushed. The force at which the strand crushes is referred to as the crushing strength of the strand. Controlling the experiment was carried out by means of a computer which registered and evaluated the results of the measurements. The values obtained are the mean value of the measurements for 10 strands in each case.

### REFERENCE EXAMPLE 7: DETERMINATION OF THE WATER UPTAKE

The water adsorption/desorption isotherms measurements were performed on a VTI SA instrument from TA Instruments following a step-isotherm program. The experiment consisted of a run or a series of runs performed on a sample material that has been placed on the microbalance pan inside of the instrument. Before the measurement was started, the residual moisture of the sample was removed by heating the sample to 100° C. (heating ramp of 5° C./min) and holding it for 6 h under a N₂ flow. After the drying program, the temperature in the cell was decreased to 25° C. and kept isothermal during the measurements. The microbalance was calibrated, and the weight of the dried sample was balanced (maximum mass deviation 0.01 weight-%). Water uptake by the sample was measured as the increase in weight over that of the dry sample. First, an adsorption curve was measured by increasing the relative humidity (RH) (expressed as weight-% water in the atmosphere inside of the cell) to which the samples was exposed and measuring the water uptake by the sample at equilibrium. The RH was increased with a step of 10% from 5% to 85% and at each step the system controlled the RH and monitored the sample weight until reaching the equilibrium conditions and recording the weight uptake. The total adsorbed water amount by the sample was taken after the sample was exposed to the 85% RH. During the desorption measurement the RH was decreased from 85% to 5% with a step of 10% and the change in the weight of the sample (water uptake) was monitored and recorded.

### REFERENCE EXAMPLE 8: DETERMINATION OF THE PROPYLENE OXIDE ACTIVITY AND THE PRESSURE DROP RATE (PO TEST)

The PO test as disclosed in the following represents a preliminary test procedure to assess the possible suitability of the moldings as catalyst for the epoxidation of propene. In the PO test, a molding is tested as catalyst in a mini autoclave with respect to the reaction of propene with hydrogen peroxide, provided as an aqueous hydrogen peroxide solution (30 weight-%) to yield propylene oxide. In particular, 0.63 g of a molding is introduced together with 79.2 g of acetonitrile and 12.4 g of propene at room temperature, and 22.1 g of the aqueous hydrogen peroxide in a steel autoclave. After a reaction time of 4 hours at 40° C., the mixture was cooled and depressurized, and the liquid phase was analyzed by gas chromatography with respect to its propylene oxide content. The propylene oxide content of the liquid phase (in weight-%) is the result of the PO test.

### REFERENCE EXAMPLE 9: DETERMINATION OF THE PROPYLENE OXIDE ACTIVITY IN A CONTINUOUS EPOXIDATION REACTION

Continuous epoxidation reaction was carried out as described in WO 2015/010990 A, in Reference Example 1, page 55, line 14 to page 57, line 10. The reaction temperature was set to a value of 45° C. (see WO 2015/010990 A, page 56, lines 16 to 18). The temperature was adjusted to achieve an essentially constant hydrogen peroxide conversion of 90% (see WO 2015/010990 A, page 56, lines 21 to 23). KH₂PO₄ was employed as additive (see WO 2015/010990 A, page 56, lines 7 to 10), the concentration of the additive was 130 micromol per mol hydrogen peroxide. As catalysts, the catalysts according to Comparative Example 22, Reference Example 20 and Example 23 hereinbelow were employed (see WO 2015/010990 A, page 55, lines 16 to 18).

### REFERENCE EXAMPLE 10: DETERMINATION OF THE C VALUE (BET C CONSTANT)

The C value was determined by usual calculation ((slope/intercept)+1) based on the plot of the BET value 1/(V((p/p₀)−1)) against p/p₀, as known by the skilled person. p is the partial vapour pressure of adsorbate gas in equilibrium with the surface at 77.4 K (b.p. of liquid nitrogen), in Pa, p₀ is the saturated pressure of adsorbate gas, in Pa, and V is the volume of gas adsorbed at standard temperature and pressure (STP) [273.15 K and atmospheric pressure (1.013×10⁵ Pa)], in mL.

### REFERENCE EXAMPLE 11: IR MEASUREMENTS

The IR measurements were performed on a Nicolet 6700 spectrometer. The zeolitic materials were pressed into a self-supporting pellet without the use of any additives. The pellet was introduced into a high vacuum cell placed into the IR instrument. Prior to the measurement the sample was pretreated in high vacuum (10⁻⁵ mbar) for 3 h at 300° C. The spectra were collected after cooling the cell to 50° C. The spectra were recorded in the range of 4000 cm⁻¹ to 800 cm⁻¹ at a resolution of 2 cm⁻¹. The obtained spectra were represented by a plot having on the x axis the wavenumber (cm⁻¹) and on the y axis the absorbance (arbitrary units). For the quantitative determination of the peak heights and the ratio of the peak heights, a baseline correction was carried out.

### REFERENCE EXAMPLE 12: DETERMINATION OF THE TORTUOSITY PARAMETER RELATIVE TO WATER

Samples were prepared for NMR analyses by drying a small quantity (0.05-0.2 g) of catalyst at T>350° C. under vacuum overnight in NMR measurement tubes. The sample was then filled via a vacuum line with nanopure water (Millipore Advantage A10) to 90% of the pore volume of the catalyst support (determined by Hg-porosimetry). The filled sample was then flame sealed into the measurement tube and left overnight before measurement.

The NMR analyses to determine the self diffusion coefficient (D_{eff}) for water in the catalyst materials were conducted at 20° C. and 1 bar at 400 MHz 1H resonance frequency with Bruker Avance III NMR spectrometer. A Bruker Diff50 probe head was used with Bruker Great 60A gradient amplifiers. A temperature of 20° C. was maintained with water cooled gradient coils. The pulse program used for the PFG NMR self-diffusion analyses was the stimulated spin echo with pulsed field gradients according to FIG. 1b of US 20070099299 A1. For each sample, the spin echo attenuation curves were measured at different diffusion times (between 20 and 100 ms) by stepwise increase in the intensity of the field gradients (to a maximum gmax=3 T/m). The gradient pulse length was 1 ms. Spin echo attenuation curves were fitted to equation 6 of US 2007/0099299 A, by way of an example, a double logarithmic plot of data from a catalyst support at the various diffusion times used is shown in figure X. The slope of each line corresponds to a diffusion coefficient. The average diffusion coefficient, across all diffusion times, was used to calculate tortuosity for each catalyst support, according to Formula II (see Reference Example 2).

PFG NMR enables the destruction free examination of thermal molecular motion, in free gases and liquids, in macro and supra molecular solutions and of adsorbed molecules in porous systems. The principle and applications are as described in US 20070099299 A1. From the diffusion coefficient obtained by NMR according to Reference Example 4, the tortuosity factor was calculated. The tortuosity factor of a porous material is determined from the self diffusion coefficient of a probe molecule in the porous system (D_{eff}) and the self diffusion coefficient of the free liquid (D₀) according to formula II (see S. Kolitcheff, E. Jolimaitre, A. Hugon, J. Verstraete, M. Rivallan, P-L. Carrette, F. Couenne and M. Tayakout-Fayolle, Catal. Sci. Technol., 2018, 8, 4537; and F. Elwinger, P. Pourmand, and I. Furo, J. Phys. Chem. C. 2017, 121, 13757-13764):

$$τ = \frac{{D}_{0}}{{D}_{e f f}} . \qquad ( ll )$$

The free diffusion coefficient for water was taken as 2.02×10⁻⁹ m² s⁻¹ at 20° C. (see M. Holz, S. R. Heil and A. Sacco. Phys. Chem. Chem. Phys., 2000, 2, 4740-4742).

### REFERENCE EXAMPLE 12: PREPARATION OF A TI-MWW

A zeolitic material having framework structure MWW and comprising Ti (also abbreviated herein as Ti-MWW) was provided similar to a zeolitic material prepared according to Example 5, 5.1 to 5.3, of WO 2013/117536 A, page 83, line 26 to page 92, line 7. The resulting zeolitic material had a crystallinity of 89%, a BET specific surface area of 353 m²/g, a C value of −94, a Ti content of 1.5 g Ti/100 g. Further, the resulting zeolitic material displayed a water adsorption of 12 weight-%.

### REFERENCE EXAMPLE 14: PREPARATION OF A TI-MWW IMPREGNATED WITH ZN

A zeolitic material having framework structure MWW, comprising Ti, and being impregnated with Zn was provided according to Reference Example 1 of WO 2013/117536 A2 on pages 57-66.

### REFERENCE EXAMPLE 15: PREPARATION OF A TI-MWW IMPREGNATED WITH BA

1.2 g barium nitrate (Ba(NO₃)₂) were solved in 60 g deionized water in a beaker under stirring for 1 h. Then, 40.0 g of Ti-MWW according to Reference Example 12 were added to the mixture and kept for 40 h at room temperature. The resulting solids were dried in air for 5 h at 110° C. and subsequently calcined in air for 8 h at 550° C. to obtain the product. The yield was 39.6 g.

The resulting material had a Ba content of 1.6 g/100 g, a Si content of 43 g/100 g, and a Ti content of 1.5 g/100 g.

### REFERENCE EXAMPLE 16: PREPARATION OF A TI-MWW IMPREGNATED WITH BA AND ZN

1.20 g barium nitrate (Ba(NO₃)₂) and 1.64 zink nitrate (Zn(NO₃)₂.6H₂O) were solved in 60.00 g deionized water in a beaker under stirring for 1 h. Then, 40.00 g of Ti-MWW according to Reference Example 12 were added to the mixture and kept for 36 h at room temperature. The resulting solids were dried in air for 5 h at 110° C. and subsequently calcined in air for 8 h at 550° C. to obtain the product. The yield was 40.3 g.

The resulting material had a Ba content of 1.6 g/100 g, a Si content of 42 g/100 g, a Ti content of 1.5 g/100 g and a Zn content of 0.88 g/100 g.

### REFERENCE EXAMPLE 17: PREPARATION OF A TI-MWW IMPREGNATED WITH BA, ZN, AND LA

1.20 g barium nitrate (Ba(NO₃)₂), 1.64 zink nitrate (Zn(NO₃)₂.6H₂O) and 1.24 g lanthanum nitrate (La(NO₃)₃.6H₂O) were solved in 60.00 g deionized water in a beaker under stirring for 1 h. Then, 40.00 g of Ti-MWW according to Reference Example 12 were added to the mixture and kept for 36 h at room temperature. The resulting solids were dried in air for 5 h at 110° C. and subsequently calcined in air for 8 h at 550° C. to obtain the product. The yield was 40.9 g.

The resulting material had a Ba content of 1.6 g/100 g, a La content of 1.0 g/100 g, a Si content of 42 g/100 g, a Ti content of 1.5 g/100 g and a Zn content of 0.88 g/100 g.

### REFERENCE EXAMPLE 18: SHAPING OF A TI-MWW IMPREGNATED WITH ZN

30 g Ti-MWW impregnated with Zn according to Reference Example 14 and 1.92 g methyl cellulose (Walocel MW 15000 GB, Wolff Cellulosics GmbH & Co. KG, Germany) were provided in a kneader and kneaded for 5 minutes. Then, 60 mL of deionized water together with 18.75 g colloidal silica (Ludox® AS 40) were added and the mixture was further kneaded for 10 minutes. Then, 10 mL of deionized water were added and the mixture was further kneaded for 15 minutes. The total kneading time was 45 minutes.

The kneaded mass was extruded at a pressure of 120 bar(abs) to give strands having a circular cross-section with a diameter of 1.7 mm. Subsequently, the extruded strands were dried and calcined in air according to the following program:

1. heating within 40 minutes up to a temperature of 120° C.;

2. keeping the temperature of 120° C. for 6 h;

3. heating within 380 minutes to a temperature of 500° C.;

4. keeping the temperature of 500° C. for 5 h.

The resulting material had a TOC of less than 0.1 g/100 g, a Zn content of 1.1 g/100 g, a Si content of 43 g/100 g, and a Ti content of 1.9 g/100 g. The Lewis acidity was determined according to Reference Example 1, whereby the integral extinction units of the IR bands of the Lewis acid sites were determined as being 14.2, and whereby the integral extinction units of the IR band at 1490 cm⁻¹ were determined as being 0. Further, the integral extinction units of the Brønstedt acid sites were observed as being 0.23, determined according to Reference Example 1. In addition, the Lewis acid site density was determined by temperature-programmed-desorption of ammonia according to Reference Example 5. Thus, the Lewis acid site density was determined via NH₃-TPD as being 0.26 mmol/g at a temperature below 200° C., no Lewis acid sites were observed in the temperature region between 200 to 400° C., and the Lewis acid site density of 0.01 mmol/g was observed at a temperature above 500° C.

### REFERENCE EXAMPLE 19: SHAPING OF A TI-MWW IMPREGNATED WITH BA

30 g Ti-MWW impregnated with Ba according to Reference Example 15 and 1.92 g methyl cellulose (Walocel MW 15000 GB, Wolff Cellulosics GmbH & Co. KG, Germany) were provided in a kneader and kneaded for 5 minutes. Then, 60 mL of deionized water together with 18.75 g colloidal silica (Ludox® AS 40) were added and the mixture was further kneaded for 10 minutes. Then, 10 mL of deionized water were added and the mixture was further kneaded for 15 minutes. The total kneading time was 45 minutes.

The kneaded mass was extruded at a pressure of 120 bar(abs) to give strands having a circular cross-section with a diameter of 1.7 mm. Subsequently, the extruded strands were dried and calcined in air according to the following program:

1. heating within 40 minutes up to a temperature of 120° C.;

2. keeping the temperature of 120° C. for 6 h;

3. heating within 380 minutes to a temperature of 500° C.;

4. keeping the temperature of 500° C. for 5 h.

The resulting material had a TOC of less than 0.1 g/100 g, a Ba content of 1.3 g/100 g, a Si content of 43 g/100 g, and a Ti content of 1.2 g/100 g. The Lewis acidity was determined according to Reference Example 1, whereby the integral extinction units of the IR bands of the Lewis acid sites were determined as being 100.7, and whereby the integral extinction units of the IR band at 1490 cm⁻¹ at a pressure of 1 mbar were determined as being 9.77. Further, no Brønstedt acid sites were observed, determined according to Reference Example 1. In addition, the Lewis acid site density was determined by temperature-programmed-desorption of ammonia according to Reference Example 5. Thus, the Lewis acid site density was determined via NH₃-TPD as being 0.15 mmol/g at a temperature below 200° C., no Lewis acid sites were observed in the temperature region between 200 to 400° C., and the Lewis acid site density of 0.02 mmol/g was observed at a temperature above 500° C.

### REFERENCE EXAMPLE 20: SHAPING OF A TI-MWW IMPREGNATED WITH BA AND ZN

30 g Ti-MWW impregnated with Ba and Zn according to Reference Example 16 and 1.92 g methyl cellulose (Walocel MW 15000 GB, Wolff Cellulosics GmbH & Co. KG, Germany) were provided in a kneader and kneaded for 5 minutes. Then, 60 mL of deionized water together with 18.75 g colloidal silica (Ludox® AS 40) were added and the mixture was further kneaded for 10 minutes. Then, 10 mL of deionized water were added and the mixture was further kneaded for 15 minutes. The total kneading time was 45 minutes.

The kneaded mass was extruded at a pressure of 120 bar(abs) to give strands having a circular cross-section with a diameter of 1.7 mm. Subsequently, the extruded strands were dried and calcined in air according to the following program:

1. heating within 40 minutes up to a temperature of 120° C.:

2. keeping the temperature of 120° C. for 6 h;

3. heating within 380 minutes to a temperature of 500° C.;

4. keeping the temperature of 500° C. for 5 h.

The resulting material had a TOC of less than 0.1 g/100 g, a Ba content of 1.2 g/100 g, a Si content of 43 g/100 g, a Ti content of 1.2 g/100 g and a Zn content of 0.69 g/100 g. The Lewis acidity was determined according to Reference Example 1, whereby the integral extinction units of the IR bands of the Lewis acid sites were determined as being 108.9, and whereby the integral extinction units of the IR band at 1490 cm⁻¹ at a pressure of 1 mbar were determined as being 11.05. Further, no Brønstedt acid sites were observed, determined according to Reference Example 1. In addition, the Lewis acid site density was determined by temperature-programmed-desorption of ammonia according to Reference Example 5. Thus, the Lewis acid site density was determined via NH₃-TPD as being 0.23 mmol/g at a temperature below 200° C., no Lewis acid sites were observed in the temperature region between 200 to 400° C., and the Lewis acid site density of 0.02 mmol/g was observed at a temperature above 500° C.

### REFERENCE EXAMPLE 21: SHAPING OF A TI-MWW IMPREGNATED WITH BA, ZN AND LA

30 g Ti-MWW impregnated with Ba, Zn and La according to Reference Example 17 and 1.92 g methyl cellulose (Walocel MW 15000 GB, Wolff Cellulosics GmbH & Co. KG, Germany) were provided in a kneader and kneaded for 5 minutes. Then, 60 mL of deionized water together with 18.75 g colloidal silica (Ludox® AS 40) were added and the mixture was further kneaded for 10 minutes. Then, 10 mL of deionized water were added and the mixture was further kneaded for 15 minutes. The total kneading time was 45 minutes.

The kneaded mass was extruded at a pressure of 120 bar(abs) to give strands having a circular cross-section with a diameter of 1.7 mm. Subsequently, the extruded strands were dried and calcined in air according to the following program:

1. heating within 40 minutes up to a temperature of 120° C.;

2. keeping the temperature of 120° C. for 6 h;

3. heating within 380 minutes to a temperature of 500° C.;

4. keeping the temperature of 500° C. for 5 h.

The resulting material had a TOC of less than 0.1 g/100 g, a Ba content of 1.2 g/100 g, a La content of 0.78 g/100 g, a Si content of 42 g/100 g, a Ti content of 1.2 g/100 g and a Zn content of 0.68 g/100 g. The Lewis acidity was determined according to Reference Example 1, whereby the integral extinction units of the IR bands of the Lewis acid sites were determined as being 118.3, and whereby the integral extinction units of the IR band at 1490 cm⁻¹ at a pressure of 1 mbar were determined as being 11.53. Further, no Brønstedt acid sites were observed, determined according to Reference Example 1. In addition, the Lewis acid site density was determined by temperature-programmed-desorption of ammonia according to Reference Example 5. Thus, the Lewis acid site density was determined via NH₃-TPD as being 0.23 mmol/g at a temperature below 200° C., no Lewis acid sites were observed in the temperature region between 200 to 400° C., and the Lewis acid site density of 0.01 mmol/g was observed at a temperature above 500° C.

### COMPARATIVE EXAMPLE 22: WATER TREATMENT OF A SHAPED TI-MWW IMPREGNATED WITH ZN

7 g of the strands prepared according to Reference Example 18 were mixed with 140 g deionized water. The resulting mixture was heated to a temperature of 145° C. for 8 h in an autoclave. Thereafter, the obtained water-treated strands were separated and sieved over a 0.8 mm sieve. The obtained strands were then washed with deionized water and pre-dried in a stream of nitrogen at ambient temperature. The washed and pre-dried strands were subsequently dried and calcined in air according to the following program:

1. heating within 60 minutes up to 120° C.;

2. keeping the temperature of 120° C. for 4 h;

3. heating within 165 minutes up to 450° C.;

4. keeping the temperature of 450° C. for 2 h.

The resulting material showed a BET specific surface area of 283 m²/g, had a TOC of less 0.1 g/100 g, a Zn content of 1.9 g/100 g, a Si content of 42° g/100 g, and a Ti content of 1.9 g/100 g, each determined as described hereinabove. The resulting material displayed a water uptake of 10.2 weight-%, determined as described in Reference Example 7. The crushing strength of the strands determined as described hereinabove was 19 N, and the pore volume determined as described hereinabove was 1.0 mL/g. The tortuosity parameter relative to water was observed as being 1.6, determined according to Reference Example 12 The Lewis acidity was determined according to Reference Example 1, whereby the integral extinction units of the IR bands of the Lewis acid sites were determined as being 77.8, and whereby the integral extinction units of the IR band at 1490 cm¹ at a pressure of 1 mbar were determined as being 8.1. Further, no Brønstedt acid sites were observed, determined according to Reference Example 1. In addition, the Lewis acid site density was determined by temperature-programmed-desorption of ammonia according to Reference Example 5. Thus, the Lewis acid site density was determined via NH₃-TPD as being 0.24 mmol/g at a temperature below 200° C., no Lewis acid sites were observed in the temperature region between 200 to 400° C., and the Lewis acid site density of 0.05 mmol/g was observed at a temperature above 500° C.

### EXAMPLE 23: WATER TREATMENT OF A SHAPED TI-MWW IMPREGNATED WITH BA AND ZN

21 g of the strands prepared according to Example 20 were mixed in four portions of each 7 g with 140 g deionized water per portion. The resulting mixtures were heated to a temperature of 145° C. for 8 h in an autoclave. Thereafter, the obtained water-treated strands were separated and sieved over a 0.8 mm sieve. The obtained strands were then washed with deionized water and pre-dried in a stream of nitrogen at ambient temperature. The washed and pre-dried strands were subsequently dried and calcined in air according to the following program:

1. heating within 60 minutes up to 120° C.;

2. keeping the temperature of 120° C. for 4 h;

3. heating within 165 minutes up to 450° C.;

4. keeping the temperature of 450° C. for 2 h.

The resulting material showed a BET specific surface area of 284 m²/g, had a TOC of less 0.1 g/100 g, a Ba content of 1.2 g/100 g, a Si content of 43° g/100 g, a Ti content of 1.2 g/100 g, and a Zn content of 0.7 g/100 g, each determined as described hereinabove. The resulting material displayed a water uptake of 10.4 weight-%, determined as described in Reference Example 7.

The resulting material displayed a concentration of acid sites of 0.25 at a temperature lower than 200° C., of 0 at a temperature in the range of from 200 to 400° C., and of 0.05 at a temperature higher than 500° C., determined by temperature programmed desorption of ammonia (NH₃-TPD) according to Reference Example 5. The crushing strength of the strands determined as described hereinabove was 9 N, and the pore volume determined as described hereinabove was 1.5 mL/g. The tortuosity parameter relative to water was observed as being 2.0, determined according to Reference Example 12. The Lewis acidity was determined according to Reference Example 1, whereby the integral extinction units of the IR bands of the Lewis acid sites were determined as being 78.5, and whereby the integral extinction units of the IR band at 1490 cm⁻¹ at a pressure of 1 mbar were determined as being 6.8. Further, no Brønstedt acid sites were observed, determined according to Reference Example 1. In addition, the Lewis acid site density was determined by temperature-programmed-desorption of ammonia according to Reference Example 5. Thus, the Lewis acid site density was determined via NH₃-TPD as being 0.25 mmol/g at a temperature below 200° C., no Lewis acid sites were observed in the temperature region between 200 to 400° C., and the Lewis acid site density of 0.05 mmol/g was observed at a temperature above 500° C.

### EXAMPLE 24: WATER TREATMENT OF A SHAPED TI-MWW IMPREGNATED WITH BA, ZN AND LA

21 g of the strands prepared according to Example 21 were mixed in four portions of each 7 g with 140 g deionized water per portion. The resulting mixtures were heated to a temperature of 145° C. for 8 h in an autoclave. Thereafter, the obtained water-treated strands were separated and sieved over a 0.8 mm sieve. The obtained strands were then washed with deionized water and pre-dried in a stream of nitrogen at ambient temperature. The washed and pre-dried strands were subsequently dried and calcined in air according to the following program:

1. heating within 60 minutes up to 120° C.;

2. keeping the temperature of 120° C. for 4 h;

3. heating within 165 minutes up to 450° C.;

4. keeping the temperature of 450° C. for 2 h.

The resulting material had a TOC of less 0.1 g/100 g, a Ba content of 1.2 g/100 g, a La content of 0.75 g/100 g, a Si content of 42° g/100 g, a Ti content of 1.1 g/100 g, and a Zn content of 0.68 g/100 g, each determined as described hereinabove. The resulting material showed a BET specific surface area of 334 m²/g. The pore volume determined as described hereinabove was 1.7 mL/g. The tortuosity parameter relative to water was observed as being 2.0, determined according to Reference Example 12. The resulting material displayed a water uptake of 11.5 weight-%, determined as described in Reference Example 7. The Lewis acidity was determined according to Reference Example 1, whereby the integral extinction units of the IR bands of the Lewis acid sites were determined as being 9.95, and whereby the integral extinction units of the IR band at 1490 cm⁻¹ at a pressure of 1 mbar were determined as being 1.6. Further, no Brønstedt acid sites were observed, determined according to Reference Example 1. In addition, the Lewis acid site density was determined by temperature-programmed-desorption of ammonia according to Reference Example 5. Thus, the Lewis acid site density was determined via NH₃-TPD as being 0.19 mmol/g at a temperature below 200° C., no Lewis acid sites were observed in the temperature region between 200 to 400° C., and the Lewis acid site density of 0.02 mmol/g was observed at a temperature above 500° C.

### EXAMPLE 25: CATALYTIC TESTINGS

### Example 25.1: Preliminary Test—PO Test

Moldings of the examples were preliminarily tested with respect to their general suitability as epoxidation catalysts according to the PO test as described in Reference Example 8. The respective resulting values of the propylene oxide activity are shown in Table 2 below.

TABLE 2
Results for catalytic testing according to Reference Example 8

| | | Integral | Integral |
|---|---|---|---|
| Molding | propylene | extinction units | extinction units |
| according | oxide | of Lewis acid | of band at |
| to # | activity/% | bands | 1490 cm⁻¹ |
| Ref. Ex. 14 | 7.88 | 56.2 | 6 |
| Ref. Ex. 15 | 7.96 | 24.8 | 3.9 |
| Ref. Ex. 16 | 8.34 | 27.5 | 4.3 |
| Ref. Ex. 17 | 8.9 | 14.2 | |
| Ref. Ex. 18 | 6.1 | 100.7 | 9.77 |
| Ref. Ex. 19 | 6.47 | 108.9 | 11.05 |
| Ref. Ex. 20 | 6.99 | 118.3 | 11.53 |
| Comp. | 9.5 | 77.8 | 8.1 |
| Example 22 | | | |

Obviously, the molding according to Comparative Example 22 exhibits a very good propylene oxide activity according to the PO test. Therefore, it can be expected that also the moldings according to the present invention are promising candidates for catalysts in industrial continuous epoxidation reactions.

### Example 25.2: Continuous Epoxidation of Propylene

a) Results for Comparative example 22, as shown in FIG. 1
The conversion was observed to be about 99% for the first 200 hours of the testing time, then dropped to about 95% at around 400 hours, and then increased again to about 99% before decreasing within about 1500 hours to about 86%. After reaching a maximum of about 98% conversion for about 50 hours after 2000 hours the conversion then decreased below 84%. The selectivity towards propylene oxide based on H₂O₂ was in a range of from about 97 to about 99% over the whole run time. The selectivity towards propylene oxide based on propene (C3) was in the range of from about 99% to almost 100% over the whole run time. The temperature was in a range of from about 32 to about 37° C. over the whole run time.

b) Results for Reference example 20, as shown in FIG. 2
The total run time was about 500 hours. The conversion was observed to be in the range of from about 87 to 96%, reaching the maximum after about 320 hours and the minimum after about 50 hours and also after about 360 hours. The selectivity towards propylene oxide based on H₂O₂ was in a range of from about 97 to about 98% over the whole run time. The selectivity towards propylene oxide based on propene (C3) was in the range of from about 97 to about 99% over the whole run time. The temperature increased from about 35 to about 44° C. within the whole run time.

c) Results for Example 23, as shown in FIG. 3
The total run time was about 900 hours. The conversion was observed to be at least 92% over the whole run time, whereby the conversion was about 99% for about the first 250 hours, then decreased slowly to a minimum of 92% before increasing again. The selectivity towards propylene oxide based on H₂O₂ was about 99% over the whole run time. The selectivity towards propylene oxide based on propene (C3) was in the range of from about 99% to almost 100% over the whole run time. The temperature was about 35° C. over the whole run time.
In summary, the molding of the present invention is especially suitable in industrial-scale processes as regards the continuous epoxidation reaction of propene and, thus, interesting for commercial purposes, since it has convincingly been shown that the molding of the present invention according to Example 23 is an ideal catalyst, allowing, at a constantly high conversion of at least 92%, for excellent selectivities with regard to propylene oxide, in particular with regard to propylene oxide based on propene.
In particular, it has been shown that compared to a molding representing the prior art according to Reference Example 20 as discussed under item b) hereinabove, the molding of the present invention showed a conversion of at least 92%, whereas the conversion observed for Reference Example 20 was in the range of from about 87 to 96%, not to mention that a higher temperature was necessary to achieve said result. Further, the selectivity based on H₂O₂ as well as based on propene was higher for the inventive molding over the whole run time.
Similarly, the molding according to Example 23 showed an improved conversion within the first about 250 hours of the testing at a high level of about 99%, whereas the molding according to Comparative Example 22 as discussed under item a) hereinabove showed a conversion that is decreasing especially within a run time of 200 to 250 hours.

### BRIEF DESCRIPTION OF FIGURES

FIG. 1: shows the results of the continuous epoxidation reaction according to Reference Example 9 for the molding of Comparative Example 22 in terms of the valuable product propylene oxide and the hydrogen peroxide conversion. The selectivity S (PO) H₂O₂ in % for propylene oxide based on H₂O₂ (mid-grey graph) is defined as moles of propylene oxide formed per unit time divided by moles of H₂O₂ consumed per unit time×100. The selectivity S (PO) C3 in % for propylene oxide based on propylene (light-grey line) is defined as moles of propylene oxide formed per unit time divided by moles of propylene consumed per unit time ×100. The conversion C in % (left ordinate) of H₂O₂ is defined as moles of H₂O₂ consumed per unit time divided by moles of H₂O₂ fed to the reactor per unit time ×100. The inlet temperature T in ° C. (right ordinate) is the inlet temperature of the heat-transfer medium. The time on stream t in hours is given on the abscissa. The starting point (t=0) is taken as the time at which the H₂O₂ metering pump is started (all other pumps are started earlier).

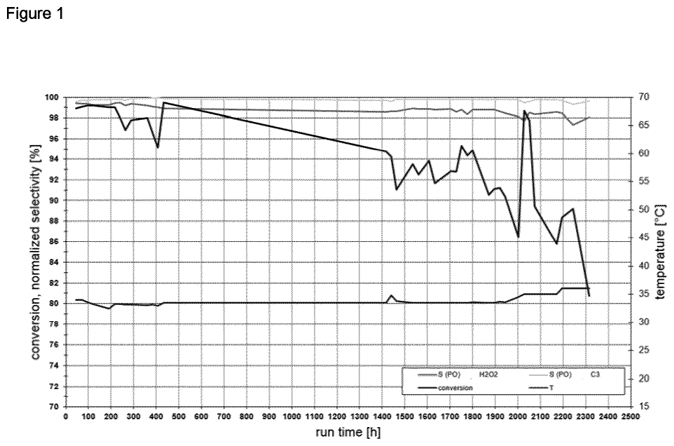

FIG. 2: shows the results of the continuous epoxidation reaction according to Reference Example 9 for the molding of Reference Example 20 in terms of the valuable product propylene oxide and the hydrogen peroxide conversion. The selectivity S (PO) H₂O₂ in % for propylene oxide based on H₂O₂ (mid-grey graph) is defined as moles of propylene oxide formed per unit time divided by moles of H₂O₂ consumed per unit time ×100. The selectivity S (PO) C3 in % for propylene oxide based on propylene (light-grey line) is defined as moles of propylene oxide formed per unit time divided by moles of propylene consumed per unit time ×100. The conversion C in % (left ordinate) of H₂O₂ is defined as moles of H₂O₂ consumed per unit time divided by moles of H₂O₂ fed to the reactor per unit time ×100. The inlet temperature T in ° C. (right ordinate) is the inlet temperature of the heat-transfer medium. The time on stream t in hours is given on the abscissa. The starting point (t=0) is taken as the time at which the H₂O₂ metering pump is started (all other pumps are started earlier).

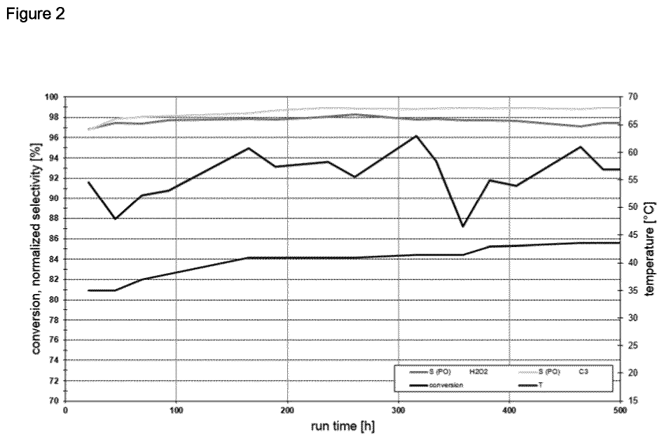

FIG. 3: shows the results of the continuous epoxidation reaction according to Reference Example 9 for the molding of Example 23 in terms of the valuable product propylene oxide and the hydrogen peroxide conversion. The selectivity S (PO) H₂O₂ in % for propylene oxide based on H₂O₂ (mid-grey graph) is defined as moles of propylene oxide formed per unit time divided by moles of H₂O₂ consumed per unit time ×100. The selectivity S (PO) C3 in % for propylene oxide based on propylene (light-grey line) is defined as moles of propylene oxide formed per unit time divided by moles of propylene consumed per unit time ×100. The conversion C in % (left ordinate) of H₂O₂ is defined as moles of H₂O₂ consumed per unit time divided by moles of H₂O₂ fed to the reactor per unit time ×100. The inlet temperature T in ° C. (right ordinate) is the inlet temperature of the heat-transfer medium. The time on stream t in hours is given on the abscissa. The starting point (t=0) is taken as the time at which the H₂O₂ metering pump is started (all other pumps are started earlier).

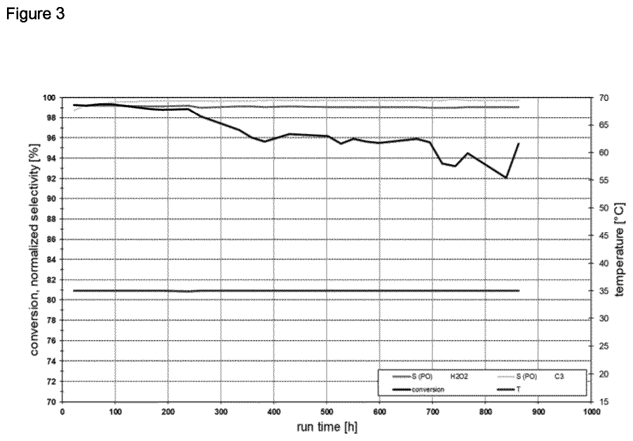

### CITED LITERATURE

CN 105854933 A
CN 106115732 A
Y. Yu et al. “Insights into the efficiency of hydrogen peroxide utilization over titanosilicate/H₂O₂ systems” in Journal of Catalysis 2020, vol. 381, p. 96-107

1.-15. (canceled)

16. A molding comprising a zeolitic material having framework type MWW, having a framework structure comprising Ti, Si, and O, wherein the zeolitic material further comprises Zn and an alkaline earth metal M, the molding further comprising a binder, wherein the molding exhibits a integral extinction units of the IR band at 1490 cm⁻¹ of equal to or smaller than 8, determined as described in Reference Example 1.

17. The molding of claim 16, comprising Si, calculated as element, in an amount in the range of from 20 to 60 weight-%, based on the total weight of the molding.

18. The molding of claim 16, comprising Ti, calculated as element, in an amount in the range of from 0.1 to 5 weight-%,

19. The molding of claim 16, comprising Zn, calculated as element, in an amount in the range of from 0.1 to 5 weight-%, based on the total weight of the molding.

20. The molding of claim 16, comprising the alkaline earth metal M, calculated as element, in an amount in the range of from 0.1 to 5 weight-%, based on the total weight of the molding.

21. The molding of claim 16, wherein the zeolitic material further comprises a rare earth metal.

22. The molding of claim 16, wherein the binder comprises Si and O.

23. The molding of claim 16, wherein the molding exhibits a total pore volume in the range of from 0.5 to 3.0 mL/g, wherein the pore volume is determined according to DIN 66133.

24. The molding of claim 16, wherein the molding comprises a concentration of acid sites in the range of from 0.05 to 1.00 mmol/g at a temperature lower than 200° C., and/or wherein the molding comprises a concentration of acid sites in the range of from 0.001 to 0.5 mmol/g at a temperature higher than 500° C., wherein the concentration of acid sites is determined by temperature programmed desorption of ammonia (NH₃-TPD) according to Reference Example 5.

25. A process for preparing a molding comprising a zeolitic material having framework type MWW and a binder material, the process comprising
i) providing a molding comprising a zeolitic material having framework type MWW, having a framework structure comprising Ti, Si, and O, wherein the zeolitic material further comprises Zn, an alkaline earth metal M, and optionally a rare earth metal, wherein the molding further comprises a binder for said zeolitic material;
ii) preparing a mixture comprising the precursor molding according to (i) and water, and subjecting the mixture to a water treatment under hydrothermal conditions, obtaining a water-treated molding, and calcining the water-treated molding in a gas atmosphere.

26. The process of claim 25, wherein (i) comprises
(i.1) providing a zeolitic material having framework type MWW and having a framework structure comprising Ti, Si, and O;
(i.2) providing an aqueous solution of a source of Zn;
(i.3) providing an aqueous solution of a source of an alkaline earth metal M;
(i.4) optionally providing an aqueous solution of a source of a rare earth metal;
(i.5) impregnating the zeolitic material provided according to (i.1) with the aqueous solution provided according to (i.2), the aqueous solution according to (i.3), and optionally the aqueous solution provided according to (i.4), obtaining an impregnated zeolitic material;
(i.6) preparing a mixture comprising the impregnated zeolitic material obtained from (i.5) and a binder precursor;
(i.7) shaping of the mixture obtained from (i.6).

27. A molding comprising a zeolitic material having framework type MWW and a binder material, obtainable or obtained by a process according to claim 25.

28. An adsorbent, an absorbent, a catalyst, or a catalyst compound comprising the molding according to claim 16.

29. A process for oxidizing an organic compound comprising bringing the organic compound in contact with a catalyst comprising a molding according to claim 16.

30. A process for preparing propylene oxide comprising reacting propene with hydrogen peroxide in acetonitrile solution in the presence of a catalyst comprising a molding according to claim 16 to obtain propylene oxide.
